SEQUENCE LISTING (10 sequences)

SEQ ID NO: 1 - DNA, 1346 bp, synthetic construct
  misc_feature 1..1346: note = promoteur IL7RA court
  source 1..1346: mol_type = other DNA
atacctaggcactaatttagttccatatgtactatgtgtacctgaaaagttgtgtggcaatcaaattttcacaaatagaatcctgttttaaatacactaagaaagtacctactttatcctttaaacaagaggtcagcagactttttctacaaagggtcagatagtaaagattttacaccttttgtacaatacaatctctatctcatctacttagctctgccattgttgcataaaagcagctgtagatgatacacaaatgggtgaggctgtattccaatgaaacgttatttgcaaaaacaggtggtagattaaatttggtcccaaggcttacttgggaaaaaaaaagatcttttgaaaaagaaaaaataaatgaataattttttaaaaaattgttccctaggtcatagtttgccagcccctgccctaaacaaataattcttgaatgcctactgtggtgtgtaagatatgagtaaataccagggatacacagagaacaaaagagaaaaactgctattcttgtgaaacttggaagttggaggtaagctatttaaaataaacccacaataaagtacttcacatagtgcagactgtttctttaaatcaaaactcactccaaacaaccaattgattcactttgtaagtttgaatttttgtcttcagattcttttaaagtgggcccttagtcaggagcggtggctcatgcctgtagtcctagcactttgggaggctgaggcaggcagatcacttgaggtcaggagttcgagacaagcctggccaacatggcgaaaccccgtctccactgaaaacacaaaaattaggctggcatagtggcatttgcctgtagtcctagctactcaggaggctgaggcaggagaattgcttgaacctgggaggtgaaaattgcagtgagccgagatcatgctattgtactccagcctgggcaacaaagcaagactctgtctcaaaaaaataaaaattaaaaaaataaagtagcctctagcctaagatagcttgagcctaggtgtgaatctactgccttactctgatgtaagcacagtaagtgtgggggctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcaga

SEQ ID NO: 2 - DNA, 1889 bp, synthetic construct
  misc_feature 1..1889: note = promoteur IL7RA long
  source 1..1889: mol_type = other DNA
tacccccattgtctgggtagttcataccagtccctcaagactagcctcaggcattgcctcttctgggaatacatcctcttacaggccaggatatgactcatgggtgcattcctaatagcacttcacttatttctactgtcaccacactgatctgtaattacttgatttgtctgactcttctgggggcttgtaagcattctggcacagagaactatgacttactggggcttacatctcttgctaaacacagtacctaaaatttagtaggcattccctcataaacatgaatgaatgaatcaaagaatgaataaacatttaggaaatgatgttgtgttggtcaacttctttcctcatcactgttaaagataaaagaatgccaagccaggttgttcagacagaagcaagcaccacatccctgagagagcagcacatctgggcagccatgtgtgagaagtcggttgcattccccatacacagttgtctttgcagctgtactcttaaccactgtaaccacagaagtggggaaacaatagggtggggtgaatacctaggcactaatttagttccatatgtactatgtgtacctgaaaagttgtgtggcaatcaaattttcacaaatagaatcctgttttaaatacactaagaaagtacctactttatcctttaaacaagaggtcagcagactttttctacaaagggtcagatagtaaagattttacaccttttgtacaatacaatctctatctcatctacttagctctgccattgttgcataaaagcagctgtagatgatacacaaatgggtgaggctgtattccaatgaaacgttatttgcaaaaacaggtggtagattaaatttggtcccaaggcttacttgggaaaaaaaaagatcttttgaaaaagaaaaaataaatgaataattttttaaaaaattgttccctaggtcatagtttgccagcccctgccctaaacaaataattcttgaatgcctactgtggtgtgtaagatatgagtaaataccagggatacacagagaacaaaagagaaaaactgctattcttgtgaaacttggaagttggaggtaagctatttaaaataaacccacaataaagtacttcacatagtgcagactgtttctttaaatcaaaactcactccaaacaaccaattgattcactttgtaagtttgaatttttgtcttcagattcttttaaagtgggcccttagtcaggagcggtggctcatgcctgtagtcctagcactttgggaggctgaggcaggcagatcacttgaggtcaggagttcgagacaagcctggccaacatggcgaaaccccgtctccactgaaaacacaaaaattaggctggcatagtggcatttgcctgtagtcctagctactcaggaggctgaggcaggagaattgcttgaacctgggaggtgaaaattgcagtgagccgagatcatgctattgtactccagcctgggcaacaaagcaagactctgtctcaaaaaaataaaaattaaaaaaataaagtagcctctagcctaagatagcttgagcctaggtgtgaatctactgccttactctgatgtaagcacagtaagtgtgggggctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcaga

SEQ ID NO: 3 - DNA, 5843 bp, synthetic construct
  misc_feature 1..5843: note = promoteur BCL11B
  source 1..5843: mol_type = other DNA
gcgcgcgggccccggcgccccgcacctgtacagtggaaccgttgccaaccgagagaatgctgacctgacttgcctccgtgtcaccgccaccccgcaccccgcgtgtccccggggcccaggggaggcggcactccaacctaacctgtgtctgcgaagtcctatggaaacccgagggttgattaaggcagtacaaattgtggagccttttaactgtgcaataatttctgtatttattgggttttgtaatttttttggcatgtgcaggtactttttattattattttttctgtttgaattcctttaagagattttgttgggtatccatcccttctttgttttttttttaacccggtagtagcctgagcaatgactcgcaagcaatgttagaggggaagcatatcttttaaattataatttggggggaggggtggtgctgcttttttgaaatttaagctaagcatgtgtaatttcttgtgaagaagccaacactcaaatgacttttaaagttgtttactttttcattccttccttttttttgtcctgaaataaaaagtggcatgcagttttttttttaattattttttaattttttttttggtttttgtttttggggtggggggtgtggatgtacagcggataacaatctttcaagtcgtagcactttgtttcagaactggaatggagatgtagcactcatgtcgtcccgagtcaagcggccttttctgtgttgatttcggctttcatattacataagggaaaccttgagtggtggtgctgggggaggcaccccacagactcagcgccgccagagatagggtttttggagggctcctctgggaaatggcccgacagcattctgaggttgtgcatgaccagcagatactatcctgttggtgtgccctggggtgccatggctgctattcgctgtagattaggctacataaaatgggctgagggtacctttttggggagatggggtggcctgcagtgacacagaaaggaagaaactagcggtgttcttttaggcgttttctggcttgacggcttctctctttttttaaatcacccccaccacataaatctcaaatcctatgttgctacaaggggtcatccatcatttcccaagcagacgaatgccctaattaattgaagttagtgttctctcatttaatgcacactgatgatattgtagggatgggtggggtggggatcttgcaaatttctattctcttttactgaaaaagcaggggatgagttccatcagaaggtgcccagcgctacttcccaggtttttattttttttttcctatctcattaggttggaaggtactaaatattgaactgttaagattagacatttgaattctgttgacccgcactttaaagcttttgtttgcatttaaattaaatggcttctaaacaagaaattgcagcatattcttctctttggcccagaggtgggttaaactgtaagggacagctgagattgagtgtcagtattgctaagcgtggcattcacaatactggcactataaagaacaaaataaaataataatttataggacagtttttctactgccattcaatttgatgtgagtgccttgaaaactgatcttcctatttgagtctcttgagacaaatgcaaaactttttttttgaaatgaaaagactttttaaaaaagtaaaacaagaaaagtacattctttagaaactaacaaagccacatttactttaagtaaaaaaaaaaaaaattctggttgaagatagaggatatgaaatgccataagacccaatcaaatgaagaaataaacccagcacaaccttggacatccattagctgaattatcctcagccccttttgtttttgggacaacgctgcttagatatggagtggaggtgatttactgctgaattaaaactcaagtgacacaagttacaagttgatatcgttgaatgaaaagcaaaacaaaaacaattcaggaacaacggctaattttttctaaagttaaatttagtgcactctgtcttaaaaatacgtttacagtattgggtacatacaagggtaaaaaaaaaattgtgtgtatgtgtgttggagcgatctttttttttcaaagtttgcttaataggttatacaaaaatgccacagtggccgcgtgtatattgttttcttttggtgacggggttttagtatatattatatatattaaaatttcttgattactgtaaaagtggaccagtatttgtaataatcgagaatgcctgggcattttacaaaacaagaaaaaaaatacccttttcttttccttgaaaatgttgcagtaaaatttaaatggtgggtctataaatttgttcttgttacagtaactgtaaagtcggagttttagtaaatttttttctgccttgggtgttgaatttttatttcaaaaaaaatgtatagaaacttgtatttggggattcaaaggggattgctacaccatgtagaaaaagtatgtagaaaaaaagtgcttaatattgttattgctttgcagaaaaaaaaaaaatcacatttctgacctgtacttatttttctcttcccgcctccctctggaatggatatattggttggttcatatgatgtaggcacttgctgtatttttactggagctcgtaattttttaactgtaagcttgtccttttaaagggatttaatgtacctttttgttagtgaatttggaaataaaaagaaaaaaaaaacaaaaacaaacaggctgccataatatatttttttaatttggcaggataaaatattgcaaaaaaaacacatttgtatgttaagtcctattgtacaggagaaaaagggttgtttgacaacctttgagaaaaagaaacaaaaggaagtagttaaatgctttggttcacaaatcatttagttgtatatattttttgtcggaattggcctacacagagaaccgttcgtgttgggcttctctctgaacgccccgaaccttgcatcaaggctccttggtgtggccacagcagaccagatgggaaattatttgtgttgagtggaaaaaaatcagtttttgtaaagatgtcagtaacattccacatcgtcctccctttctctaagaggccatctctaagatgtcagatgtagaggagagagagcgagagaacatcttccttctctaccatcactcctgtggcggtcaccaccaccacctctcccgcccttaccagcagaaagcaatgcaaactgagctgctttagtccttgagaaattgtgaaacaaacacaaatatcataaaaggagctggtgattcagctgggtccaggtgaagtgacctgctgttgagaccggtacaaattggatttcaggaaggagactccatcacagccaggacctttcgtgccatggagagtgttggcctcttgtctttcttccctgctttgctgctttgctctctgaaacctacattccgtcagtttccgaatgcgagggcctgggatgaatttggtgcctttccatatctcgttctctctccttcccctgcgtttcctctccatccttcatcctccattggtcctttttttttctttcattttttatttaatttcttttcttcctgtctgttcctcccctaatcctctattttatttttattttttgtaaagccaagtagctttaagataaagtggtggtcttttggatgagggaataatgcatttttaaataaaataccaatatcaggaagccattttttatttcaggaaatgtaagaaaccattatttcaggttatgaaagtataaccaagcatccttttgggcaattccttaccaaatgcagaagcttttctgttcgatgcactctttcctccttgccacttacctttgcaaagttaaaaaaaaggggggagggaatgggagagaaagctgagatttcagtttcctactgcagtttcctacctgcagatccaggggctgctgttgcctttggatgccccactgaggtcctagagtgcctccagggtggtcttcctgtagtcataacagctagccagtgctcaccagcttaccagattgccaggactaagccatcccaaagcacaagcattgtgtgtctctgtgactgcagagaagagagaattttgcttctgttttgtgtttaaaaaaccaacacggaagcagatgatcccgagagagaggcctctagcatgggtgacccagccgacctcaggccggtttccgcactgccacaactttgttcaaagttgcccccaattggaacctgccacttggcattagagggtctttcatggggagagaaggagactgaattactctaagcaaaatgtgaaaagtaaggaaatcagcctttcatcccggtcctaagtaaccgtcagccgaaggtctcgtggaacacaggcaaacccgtgattttggtgctccttgtaactcagccctgcaaagcaaagtcccattgatttaagttgtttgcatttgtactggcaaggcaaaatatttttattaccttttctattacttattgtatgagcttttgttgtttacttggaggttttgtcttttactacaagtttggaactatttattattgcttggtatttgtgctctgtttaagaaacaggcacttttttttattatggataaaatgttgagatgacaggaggtcatttcaatatggcttagtaaaatatttattgttcctttattctctgtacaagattttgggcctctttttttccttaatgtcacaatgttgagttcagcatgtgtctgccatttcatttgtacgcttgttcaaaaccaagtttgttctggtttcaagttataaaaataaattggacatttaacttgatctccaaaagccatagagagaccgagagctcccagagaacccggactccgccatcttcacgttgcaatctatagctcccagtctgcgcccgcaccgacccaggcgcactgggcgagccgcccctccgccccgctccccccgggcccgcggcgccaggggagcgctggggagcgctcgcggggactcggcagcccctccggccggcgccagcctgccaggtgaactgggaaggagcctctcctggtgtccccccagagcctcggaagggtcagtctccgagaaaaaacaaaaacaaaaacaaaaacaggcagcccgcagaaaacccagaaacaaccgaaccggggcagttttacattgctttgggttttttgagggggcggggtgagggggtacgagacaagtccccaagttttctttgcttttttttttcctttgctttttcttcttttatttttttatttgtttgcattttttttcccctcctggtagaagtgcgctttccacctaccagaccctgaaagaaagtgtcaggagccggtgcaaaacccagtttaagttcaagaagacatttgcaagtgcaagaggccaagcagtttgaagaagtgtaagagattttttttccttcgaaagaatatatttttaaagaaaccagccagtccgcggaaagcaacagcagttttttttttttttgcctctttttcttattttagatcgagaggtttttcttgcttttcttcccttttttttctttttgcaaacaaaacaaaaaacagcatagaagaaagagcaaaataaagaagaagaagaggaggaagagagggaaagagaggaagggaaaaaaaacaccaacccgggcagaggaggaggtgcggcggcggcggcggcggcggcagcggcggcagcggcgcggcggcggctcggaccccctcccccggctccccccatcagtgcagctctccgggcgatgccagaatagatgccggggca

SEQ ID NO: 4 - DNA, 20 bp, synthetic construct
  misc_feature 1..20: note = primer 1
  source 1..20: mol_type = other DNA
tcgctctgttggtcatcttg

SEQ ID NO: 5 - DNA, 20 bp, synthetic construct
  misc_feature 1..20: note = primer 2
  source 1..20: mol_type = other DNA
ggagactgggccatacgata

SEQ ID NO: 6 - DNA, 22 bp, synthetic construct
  misc_feature 1..22: note = primer 3
  source 1..22: mol_type = other DNA
ggcagaccgagatgaatcctca

SEQ ID NO: 7 - DNA, 21 bp, synthetic construct
  misc_feature 1..21: note = primer 4
  source 1..21: mol_type = other DNA
caggtccaggggtcttggtcc

SEQ ID NO: 8 - DNA, 20 bp, synthetic construct
  misc_feature 1..20: note = primer 5
  source 1..20: mol_type = other DNA
aaaaattcctcgtccccggt

SEQ ID NO: 9 - DNA, 22 bp, synthetic construct
  misc_feature 1..22: note = primer 6
  source 1..22: mol_type = other DNA
ggctttgatgactttctgtgct

SEQ ID NO: 10 - DNA, 12 bp, synthetic construct
  source 1..12: mol_type = other DNA
taaatcacwgca

(19) United States
(12) Patent Application Publication
(10) Pub. No.: US 2025/0049851 A1
(43) Pub. Date: Feb. 13, 2025

(54) T CELL PROGENITOR HAVING CONTROLLED EXPRESSION OF A TRANSGENE OF INTEREST

(71) Applicants: UNIVERSITE CLAUDE BERNARD LYON 1, Villeurbanne (FR); CENTRE NATIONAL DE LA RECHERCHE SCIENTIFIQUE, Paris (FR); INSERM (INSTITUT NATIONAL DE LA SANTE ET DE LA RECHERCHE MEDICALE), Paris (FR); CENTRE LEON BERARD, Lyon (FR)

(72) Inventor: Inna MENKOVA, Lyon (FR)

(21) Appl. No.: 18/721,954

(22) PCT Filed: Dec. 21, 2022
(86) PCT No.: PCT/FR2022/052469

§ 371 Date: Jun. 20, 2024

(30) Foreign Application Priority Data
Dec. 21, 2021 (FR) FR2114168

(51) Int. Cl. A61K 35/17; A61K 39/00; C12N 5/0783; C12N 15/86

(52) U.S. Cl. CPC A61K 35/17; A61K 39/4611; A61K 39/4631; A61K 39/464412; C12N 5/0636; C12N 15/86; A61K 2239/13; C12N 2501/125; C12N 2501/145; C12N 2501/2307; C12N 2501/26; C12N 2501/42; C12N 2506/11; C12N 2510/00; C12N 2740/15043

(57) ABSTRACT

The invention relates to a progenitor T cell comprising an expression cassette that enables controlled expression of a trans gene of interest based on the stage of maturation of said progenitor T cell. The invention also relates to a cell population enriched in progenitor T cells of this kind, to a process for preparing progenitor T cells, and to the use of said cells in a treatment method.

### TECHNICAL FIELD

The invention relates to a T-cell progenitor comprising an expression cassette which allows controlled expression of a transgene of interest, depending on the stage of maturation of said T-cell progenitor. The invention also relates to a cell population enriched in such T-cell progenitors, a method for preparing the T-cell progenitors, and their use in therapy.

### TECHNOLOGICAL BACKGROUND

Targeted immunotherapies involve the use of immune cells or molecules that engage immune cells to treat various diseases, including cancers, infections, or autoimmune disorders. For example, the genetic modification of T cells to express chimeric antigen receptors that target tumour antigens has recently proved successful in the treatment of acute lymphoblastic leukaemia and non-Hodgkin's lymphomas. This involves removing T cells from the patient, modifying them in vitro to make them express the chimeric antigen receptor (CAR) and re-infusing them into the same patient. These cells can specifically recognize target molecules on the surface of cancer cells by means of chimeric receptors and mobilize the entire immune system to increase its natural response. The very high cost of the treatment (approximately €400,000/patient) and the failures of the preparation of cell batches for some patients prevent the application of this technology to all patients who need it.

Allogeneic approaches, based on the use of T-cells from healthy donors, could address these issues. Allogeneic T cells from peripheral blood of donors can be used for adoptive T-cell therapy. However, the use of mature T-cells has some disadvantages. In particular, the occurrence of graft versus host disease (GvHD) due to the repertoire of T cell receptors (TCRs) expressed by these mature cells requires genetic manipulation in order to suppress the genes responsible for rejection, for example those of the major histocompatibility complex (MHC). In addition, mature T cells rapidly differentiate into short-lived effector cells, thus limiting their therapeutic activity. The administration of modified T-cell progenitors, generated in vitro from hematopoietic stem cells, is considered to overcome these problems. Thus, unlike mature T cells, modified T-cell progenitors migrate into the host thymus and are subjected to a natural selection of their TCR repertoire allowing them to develop tolerance to host antigens. In addition, the continuous generation of mature T cells in vivo from the genetically modified T cell progenitors allows for improved cell persistence and thus improved therapeutic activity.

There is still a need to improve these cell therapy strategies. Indeed, the maturation process in the thymus is accompanied by two stages of selection before the release of T cells into the circulation: positive selection and negative selection. Positive selection makes it possible to preserve only the T cells capable of recognizing antigens in a context restricted to self MHC. Negative selection allows the destruction of T cells that attack the cells of the body (autoimmune reactions).

The generation of modified T-cell progenitors therefore requires going through these two selection steps, and in particular, preventing cell depletion during the negative selection step. Since the negative selection step is very sensitive, additional strategies for coping with depletion at this step must be exploited, for example the use of promoters which allow temporal, conditional, and therefore maturation-dependent expression.

### SUMMARY OF THE INVENTION

The inventors propose to improve these cell therapy strategies by preventing T-cell progenitors expressing the transgene of interest from being depleted during thymic selection steps, so as to allow increased availability of modified T-cells.

The present invention relates to a T-cell progenitor that expresses a nucleic acid sequence of interest in a controlled manner. More particularly, the nucleic acid sequence of interest is placed under the control of an exogenous promoter induced by the Notch pathway, chosen between the IL7RA and the BCL11B promoter, thus allowing controlled expression according to the maturation state of the T-cell progenitors. The invention aims here to provide modified T-cell progenitors, whose expression of the transgene is induced at some stages of maturation and not at others (so-called “maturation-dependent” expression), thus making it possible to not express the transgene during certain critical thymic selection steps, in order to avoid cell depletion during these stages of maturation in the thymus. The proposed system aims to restrict the expression of the transgene in cells engaged in the T lymphoid differentiation pathway, while avoiding the expression of the transgene at certain critical stages of thymic selections and in other non-T cell lines.

Thus, a first aspect of the invention relates to an isolated T-cell progenitor, preferably human, having a CD45RA+, CD7+, CD5+ phenotype, said T-cell progenitor comprising an expression cassette which comprises an exogenous promoter induced by the Notch pathway chosen between the IL7RA and the BCL11B promoter, the promoter being functionally linked to a heterologous nucleic acid sequence of interest.

In a particular embodiment, said cell has a CD45RA+, CD7+, CD5+, CD1a+ phenotype.

In a particular embodiment, the IL7RA promoter sequence does not comprise the nucleic acid sequence SEQ ID NO: 2. In another particular embodiment, the IL7RA promoter sequence comprises or consists in the nucleic acid sequence SEQ ID NO: 2.

In a particular embodiment, the heterologous nucleic acid sequence of interest encodes a chimeric antigen receptor.

The invention also relates to a cell population enriched in T-cell progenitors as defined above, said population preferably comprising at least 40%, 50%, 60%, 70%, 80%, preferably at least 90% of said T-cell progenitors.

The invention also relates to a pharmaceutical composition comprising the T-cell progenitor or cell population.

The invention also relates to an expression cassette comprising the IL7RA promoter region functionally linked to a heterologous nucleic acid sequence of interest, in which the IL7RA promoter region comprises or consists in a nucleic acid sequence having at least 85%, 90%, 95%, 96%, 97%, 98%, or at least 99% identity with the nucleic acid sequence SEQ ID NO: 1 or SEQ ID NO: 2. According to a particular embodiment, the nucleic acid sequence of interest encodes a chimeric antigen receptor or a T cell receptor.

The invention further relates to an expression cassette comprising the BCL11B promoter functionally linked to a heterologous nucleic acid sequence of interest, wherein the BCL11B promoter preferably comprises or consists of a nucleic acid sequence having at least 85%, 90%, 95%, 96%, 97%, 98%, or at least 99% identity with the nucleic acid sequence SEQ ID NO: 3. According to a particular embodiment, the nucleic acid sequence of interest encodes a chimeric antigen receptor or a T cell receptor.

The invention also relates to the use of an expression cassette comprising the IL7RA or BCL11B promoter region functionally linked to a heterologous nucleic acid sequence of interest, or of a vector comprising it, for the generation of T-cell progenitors.

Another aspect of the invention relates to a vector comprising an expression cassette as defined above. The vector is preferably a viral vector, preferably a retroviral vector, and more preferentially a lentiviral vector.

The invention also relates to a method for preparing T-cell progenitors as defined above, comprising the steps of:
a) growing CD34+ hematopoietic stem cells in a culture medium comprising cytokines for differentiating said CD34+ cells into T-cell progenitors having a CD45RA+, CD7+, CD5+ phenotype, preferably a CD45RA+, CD7+, CD5+, CD1a+ phenotype;
b) transforming or transducing cells before, during or after said differentiation step (a) with a vector comprising an expression cassette as defined above;
wherein the CD34+ hematopoietic stem cells are preferably derived from umbilical cord blood, peripheral blood or bone marrow, more preferentially umbilical cord blood.

Another aspect of the invention relates to said T-cell progenitor, said cell population or said pharmaceutical composition, for its use in the treatment of a cancer, an infectious disease, a genetic disease, an inflammatory disease or an immune or autoimmune disease. According to a particular embodiment, said T-cell progenitors are cells that are autologous or allogeneic with respect to the patient to be treated, said cells preferably being allogeneic cells.

The invention also relates to an in-vitro method for determining whether a subject is likely to respond to treatment with a pharmaceutical composition of the invention, said method comprising:
a) measuring the quantity of recent thymic emigrant (RTE) cells in the sample,
b) comparing the quantity of RTE cells measured in the sample with a reference value,
c) determining whether the subject is likely to respond to treatment with a pharmaceutical composition of the invention, in which a quantity of RTE cells equal to or greater than the reference value indicates a likelihood to respond to treatment.

Another aspect of the invention relates to a method of treating a disease or disorder in a subject comprising:
a) determining whether the subject is likely to respond to such treatment by in-vitro evaluation of thymic activity in a blood sample of the subject, in particular by:
   measuring the quantity of recent thymic emigrant (RTE) cells in the sample,
   comparing the quantity of RTE cells measured in the sample with a reference value,
   determining whether the subject is likely to respond to treatment with a pharmaceutical composition of the invention, in which a quantity of RTE cells equal to or greater than the reference value indicates a likelihood to respond to treatment.
b) administering to the subject likely to respond to treatment a therapeutically effective quantity of a pharmaceutical composition of the present invention.

### BRIEF DESCRIPTION OF THE FIGURES

### [FIG. 1]. Schematic of the IL7RA Promoter

The IL7RA promoter is considered to be located in positions −1257 to +89 of the gene relative to the transcription initiation site. Transcription factors “GFi-1” and “GABPa” bind to 5′-TAAATCAC[AT]GCA-3′ sequences (SEQ ID NO: 10) and purine-rich repeats (GA repeats) respectively. The promoter contains CpG methylation islands in positions −975, −567, −497, −475, −466 and −346. The promoter comprises the TATA box, in position −27 to −22 of the human IL7RA gene. The start codon “ATG” is in position +90.

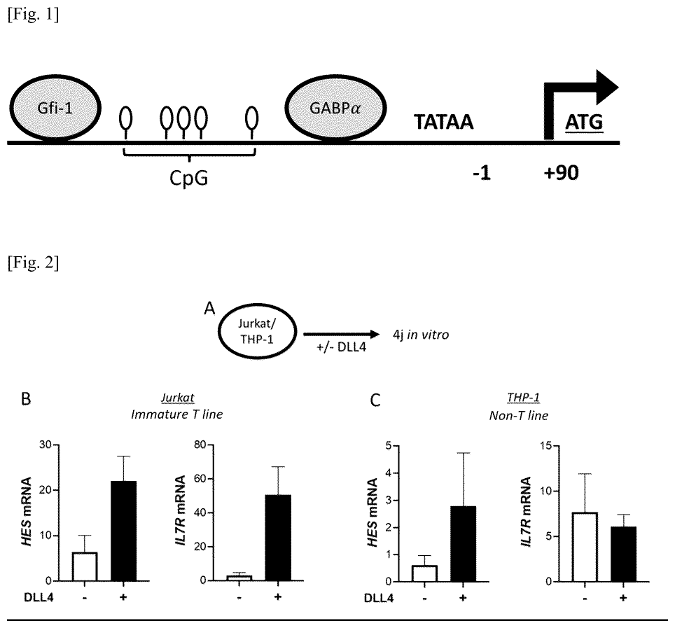

### [FIG. 2]. Expression of IL7RA Following Activation of the Notch Pathway in Lymphoid and Non-Lymphoid Cells

FIG. 2-A: schematic representation of the protocol consisting of growing Jurkat (lymphoid) and THP-1 (non-lymphoid) cells in the presence or absence of a Notch ligand (Delta-like 4, DLL4) for 4 days.

FIG. 2-B: molecular analysis of IL7RA and HES (control) mRNA expression by RT-qPCR in Jurkat cells grown in the presence (+) or absence (−) of DLL4.

FIG. 2-C: molecular analysis of IL7RA and HES (control) mRNA expression by RT-qPCR, in THP-1 cells (non-T lineage), grown in the presence (+) or absence (−) of DLL4.

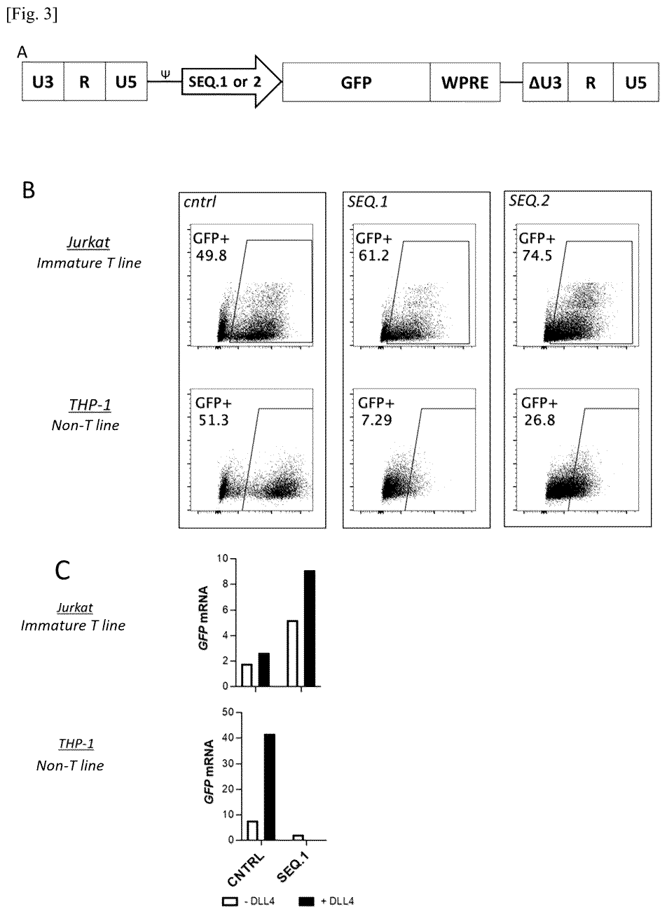

### [FIG. 3]. Expression of the GFP Transgene Under the Control of the IL7RA Promoter

FIG. 3-A: schematic representation of the cassette comprising the restricted IL7RA promoter sequence (SEQ ID NO: 1, “SEQ. 1”) or the extended sequence (SEQ ID NO: 2, “SEQ. 2”) functionally linked to GFP, and cloned into a lentiviral vector. The lentiviral vector is used to transduce Jurkat and THP-1 cells.

FIG. 3-B: analysis of GFP expression by flow cytometry in transduced Jurkat or THP-1 cells, depending on the promoter used: (i) control promoter (“cntrl”, corresponding to the spleen focus-forming virus “SFFV” promoter), (ii) restricted sequence (SEQ. 1) of the IL7RA promoter or (iii) extended sequence (SEQ. 2) of the IL7RA promoter.

FIG. 3-C: molecular analysis of GFP mRNA expression by RT-qPCR, in transduced Jurkat or THP-1 cells, in the presence (+) or absence (−) of DLL4 depending on the promoter used: (i) control promoter (“cntrl”), or (ii) restricted sequence (SEQ. 1) of the IL7RA promoter.

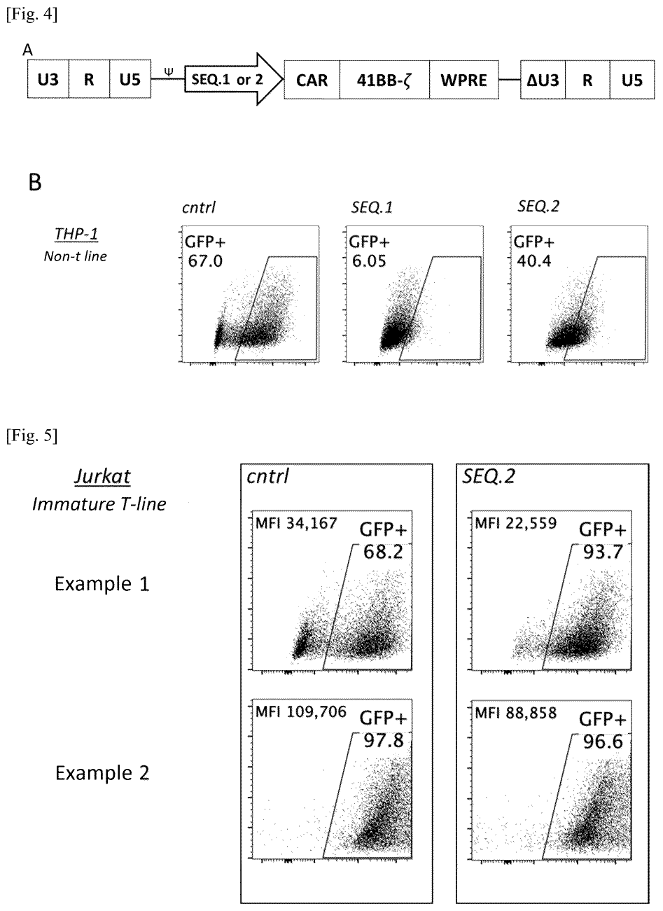

### [FIG. 4]. Expression of the CAR Transgene Under the Control of the IL7RA Promoter

FIG. 4-A: schematic representation of the cassette comprising the restricted sequence of the IL7RA promoter (SEQ ID NO: 1, “SEQ 1”) or extended sequence (SEQ ID NO: 2, “SEQ. 2”) functionally linked to the sequence encoding a chimeric antigen receptor (CAR-GFP), and cloned into a lentiviral vector. The lentiviral vector is used to transduce THP-1 cells.

FIG. 4-B: analysis of GFP expression in transduced THP-1 cells, by flow cytometry, depending on the promoter used: (i) control promoter (SFFV promoter, “cntrl”), (ii) restricted sequence (SEQ. 1) of the IL7RA promoter or (iii) extended sequence (SEQ. 2) of the IL7RA promoter.

### [FIG. 5]. The IL7RA Promoter is a Strong Promoter

Analysis of GFP expression by flow cytometry (two replicas), in transduced Jurkat cells, depending on the promoter used: (i) control promoter (SFFV promoter, “cntrl”), or (ii) extended sequence (SEQ. 2) of the IL7RA promoter.

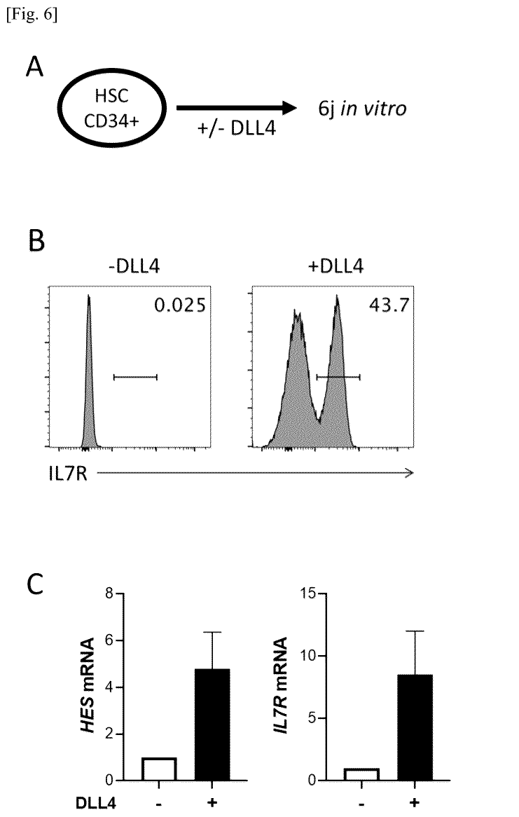

### [FIG. 6]. Expression of IL7RA is Induced Via the Notch Pathway in Haematopoietic Stem Cells

FIG. 6-A: schematic representation of the protocol consisting of growing CD34+ hematopoietic stem cells (HSC) in the presence or absence of a Notch ligand (Delta-like 4, DLL4) for 6 days.

FIG. 6-B: analysis of the expression of the endogenous IL7R protein in the presence (+) or absence (−) of DLL4 in CD34+ hematopoietic stem cells (HSC).

FIG. 6-C: molecular analysis of IL7RA and HE'S mRNA expression by RT-qPCR in hematopoietic stem cells grown in the presence (+) or absence (−) of DLL4.

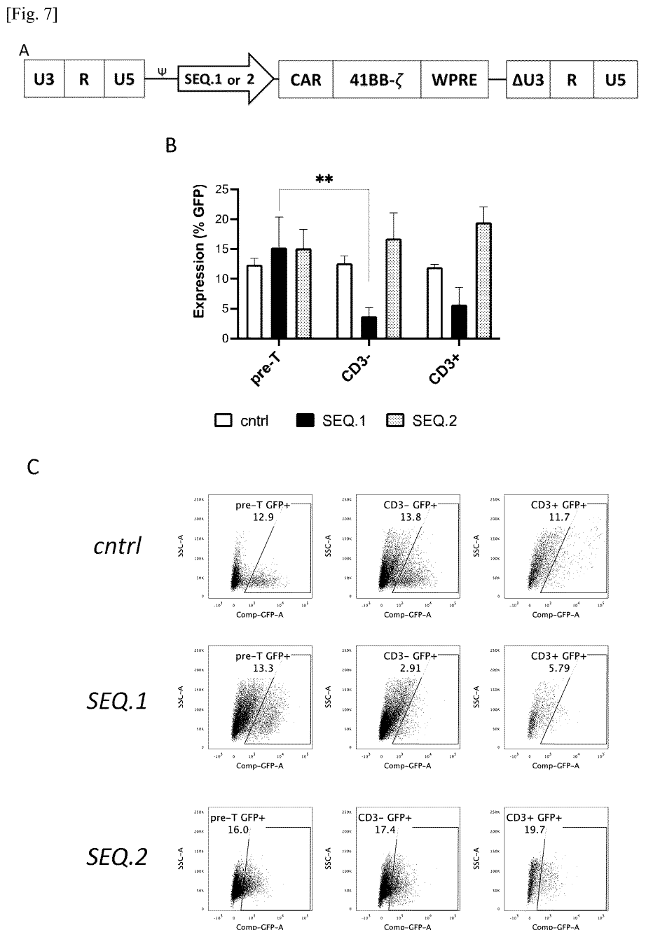

### [FIG. 7]. Transgene Expression Under IL7RA Promoter Control During T Differentiation in Vitro

FIG. 7-A: schematic representation of the cassette comprising the restricted sequence of the IL7RA promoter (SEQ ID NO: 1, “SEQ. 1”) or extended sequence (SEQ ID NO: 2, “SEQ. 2”) functionally linked to the sequence encoding a chimeric antigen receptor (CAR-GFP), and cloned into a lentiviral vector. The lentiviral vector is used to transduce CD34+ hematopoietic stem cells grown in a differentiation medium.

FIGS. 7-B and 7-C: analysis of GFP expression by flow cytometry, in different populations of interest: T precursors (CD45RA+CD7+, “pre-T”), CD3-negative (CD4−CD8−CD3−, “CD3−”) and CD3-positive (CD4−CD8−CD3+, “CD3+”), depending on the promoter used: (i) control (“cntrl”), (ii) restricted sequence (SEQ. 1) or (iii) extended sequence (SEQ. 2) of the IL7RA promoter. The mean of the 3 independent experiments is represented (B) as well as data from one of the experiments (C).

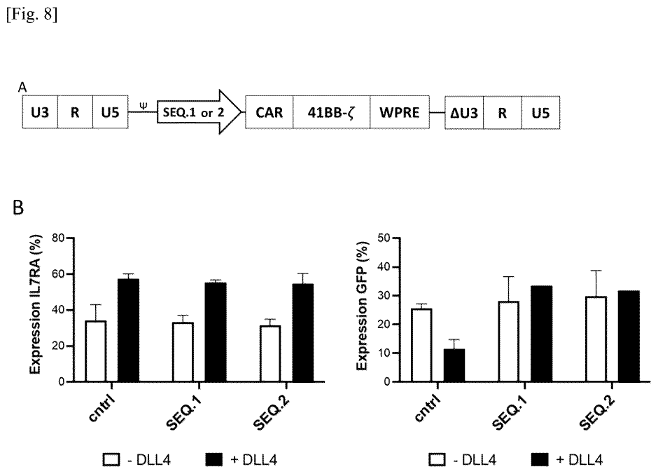

### [FIG. 8]. Expression of the Endogenous IL7RA Gene and Transgene Under IL7RA Promoter Control

FIG. 8-A: schematic representation of the cassette comprising the restricted sequence of the IL7RA promoter (SEQ ID NO: 1, “SEQ. 1”) or extended sequence (SEQ ID NO: 2, “SEQ. 2”) functionally linked to the sequence encoding a chimeric antigen receptor (CAR-GFP), and cloned into a lentiviral vector. The lentiviral vector is used to transduce naïve CD4+ T cells grown in the presence (+) or absence (−) of DLL4.

FIG. 8-B: analysis of the expression of IL7RA and of the CAR-GFP transgene by flow cytometry, in naïve CD4+ T cells grown in the presence (+) or absence (−) of DLL4, depending on the promoter used: (i) control promoter (“cntrl”), (ii) restricted sequence (SEQ. 1) of the IL7RA promoter or (iii) extended sequence (SEQ. 2) of the IL7RA promoter.

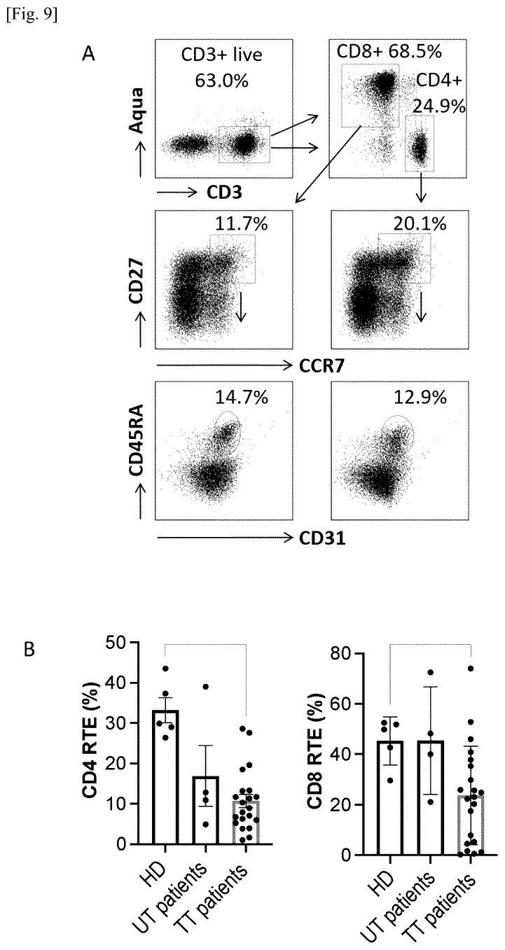

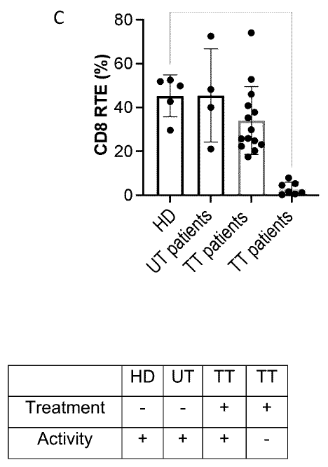

### [FIG. 9]. Measurement of Thymic Activity.

FIG. 9-A: example of labelling CD4-Pacific Blue, CD3-Alexa Fluor 700, CD8-APC-H7, CCR7-Alexa Fluor 647 (BD Biosciences), CD45RA-PE-TexasRed, CD31-PE (Miltenyi Biotech) and CD27-PE-Cy7 (eBioscience), LIVE/DEAD aqua fluorescent dye (Invitrogen) of whole blood samples.

FIG. 9-B: measurement of the percentage of CD4 RTE (left) and CD8 RTE (right) cells in whole blood samples from healthy donors (HD, n=5), untreated leukaemia or lymphoma patients (UT patients, n=4) and treated patients (TT patients, n=21).

FIG. 9-C: measurement of the percentage of CD8 RTE cells in whole blood samples from healthy donors (HD, n=5), untreated leukaemia or lymphoma patients (UT patients, n=4) and treated patients (TT patients, n=21). The group of treated patients is represented in 2 subgroups: those with thymic activity (n=14) and those without thymic activity (n=7).

### DETAILED DESCRIPTION

A first aspect of the invention relates to an isolated T-cell progenitor, modified so as to comprise an expression cassette comprising an exogenous promoter induced by the Notch pathway chosen between the IL7RA and the BCL11B promoter, said promoter being functionally linked to a heterologous nucleic sequence of interest.

The term “isolated” as used herein means that the progenitor cell is separated or isolated from its natural environment. This expression thus distinguishes isolated cells from cells forming an integral part of the human body.

Preferably, the T-cell progenitor is a mammalian cell. Preferably, the T-cell progenitor is a human cell.

The term “T-cell progenitor” or “pro-T cell” as used herein refers to a T cell that has the potential to differentiate into cells exhibiting the morphological, physiological, functional and/or immunological characteristics of T cells. In particular, the T-cell progenitor according to the invention corresponds to a cell having lost all differentiation potential except the differentiation potential toward the lymphoid pathway. T-cell progenitors can be easily characterized, based on the expression of surface antigens such as CD7, CD45RA, CD5, CD1a, CD3, CD4 and CD8. The expression of surface antigens can be easily determined, especially by flow cytometry.

According to a particular embodiment, the T-cell progenitors according to the invention have the CD45RA+, CD7+, CD5+ phenotype, preferably the CD45RA+, CD7+, CD5+, CD1a+ phenotype.

According to a particular embodiment, the T-cell progenitors according to the invention have the following phenotype:
CD45RA+, CD7+, CD5+, CD1a−, CD3−, CD4−, CD8− (T progenitors) or
CD45RA+, CD7+, CD5+, CD1a+, CD3−, CD4−, CD8− (T precursors).

Preferably, the T-cell progenitors according to the invention have the phenotype CD45RA+, CD7+, CD5+, CD1a+, CD3−, CD4−, CD8− (T precursors).

T-cell progenitors express thymic homing receptors, i.e. receptors that allow T-cells to reside in the thymus (e.g. CD5 or CXCR4, CD62L, CCR7 and CCR9), and are capable of migrating into the thymus after their injection into the peripheral blood to finish their maturation and become tolerated cells with respect to a given recipient.

In the present invention, the T-cell progenitor is genetically modified ex vivo to express a nucleic acid sequence of interest, under the control of an exogenous promoter induced by the Notch pathway chosen between the IL7RA and the BCL11B promoter.

The term “exogenous” promoter is understood to mean a promoter which is not a promoter naturally present in the T-cell progenitor. Thus, the IL7RA promoter is not the endogenous IL7RA promoter naturally present in the T-cell progenitor genome. Similarly, the BCL11B promoter according to the invention is not the endogenous BCL11B promoter naturally present in the genome of the T-cell progenitor.

The T-cell progenitor according to the invention comprises an expression cassette, which itself comprises: an exogenous promoter induced by the Notch pathway chosen between the IL7RA and the BCL11B promoter, said promoter being functionally linked to a heterologous nucleic sequence of interest.

Nucleic sequence is understood to mean any sequence consisting of nucleic acids, such as deoxyribonucleic acids or ribonucleic acids.

The term “functionally linked” or “operably linked” refers to a juxtaposition of two nucleic acid sequences in which the sequences are in a relationship that allows them to function as intended. For example, a promoter is said to be operably/functionally linked to the coding nucleic sequence, if the linkage or the connection allows transcription of said nucleic sequence. For example, DNA sequences, such as, for example, a promoter and a molecule encoding the nucleic acid, are said to be functionally linked if the nature of the linkage between the sequences (1) does not lead to the introduction of a reading frame shift, (2) does not interfere with the ability of the promoter to direct transcription of the coding nucleic region, or (3) does not interfere with the ability of the nucleic region of interest to be transcribed by the promoter region sequence.

The nucleic acid sequence of interest can be linked to other regulatory sequences, such as transcription enhancers, terminators, Kozak sequence, etc.

The term “promoter” used here means a promoter region which controls the transcription of a particular gene, and includes a promoter and/or an enhancer. A promoter is a region of DNA that initiates the transcription of a particular gene. Promoters are located close to the gene transcription initiation sites, on the same strand and upstream of the DNA (toward the 5′ region of the sense strand). Promoters generally have a length of approximately 100 to 1000 base pairs. An enhancer is a short region (50-1500 bp) of DNA that can be linked by transcription factors to stimulate gene transcription. Enhancers can be found far from the genes whose expression they control (<1,000,000 bp).

In the present invention, the promoter is a promoter induced (or activated) by the Notch pathway. A promoter “induced by the Notch pathway” is a promoter whose transcription initiation activity will be activated by the Notch pathway. The Notch protein is a receptor in its extracellular part. Upon contact with the ligand, the intracellular part of the Notch protein (ICN) is cleaved and then translocated into the nucleus of the cell where it acts as a transcription factor. Depending on the physiological context, ICN associates with other proteins, and this complex binds to promoters and/or enhancers of the target genes, favouring a dynamic context for the regulation of the target genes. The Notch protein has no transcriptional activity of its own and acts as a recruitment platform for additional co-activators.

The inventors propose to use a promoter of a target gene of the Notch pathway, in order to allow the expression of the transgene in cells engaged in the T-lymphoid differentiation pathway, while avoiding the expression of the transgene at certain stages of thymic selections. The use of such promoters advantageously makes it possible to not express the transgene during certain critical thymic selection steps, in order to avoid depletion of modified T-cell progenitors, during these maturation steps in the thymus.

The IL7RA and BCL11B promoters used in the present invention are promoters activated by the Notch pathway. In other words, the attachment of Notch ligands (for example delta-like 1, DLL-1 and delta-like 4, DLL-4) to the extracellular part of Notch leads to an increase in expression of the IL7RA and BCL11B genes.

The expression cassette of the present disclosure can comprise a promoter (or “promoter region”) derived from the IL7RA gene (also called the IL7R, IL7R-alpha or CD127 gene), preferably a promoter derived from the human IL7RA gene. The term “derived from” as used herein indicates a relationship between a first molecule and a second molecule, and generally refers to the structural similarity between the two molecules and does not require that one of them be physically generated from the other. In the present invention, a promoter “derived” from a human IL7RA gene refers to a promoter that contains a functionally active region of the promoter of a human IL7RA gene, or a functional equivalent thereof (e.g., a variant of the promoter region containing nucleotide changes relative to the native sequence where the nucleotide changes do not adversely affect the transcriptional regulatory activity of the promoter).

According to a particular embodiment, the sequence of the IL7RA promoter introduced into the expression cassette corresponds to a sequence located in the region of −1257 to +89 base pairs (relative to the transcription initiation site) of the IL7RA gene. The expression “nucleotides X to Y” of a human IL7RA gene is understood to mean nucleotides X to Y of the human IL7RA gene as referenced in the NCBI database (NCBI reference: 3575), or Ensembl database (Ensembl reference: ENSG00000168685). In the present invention, the nucleotide “+1” corresponds to the transcription initiation site. The start codon (ATG) of the human IL7RA gene is thus in position +90 of exon 1.

According to a particular embodiment, the sequence of the IL7RA promoter introduced into the expression cassette comprises the potential Notch binding domains of the native IL7RA promoter (nucleotides −927 to −920). Thus, according to a particular embodiment, the IL7RA promoter used in the expression cassette comprises nucleotides −927 to −920 of the human IL7RA gene.

According to a particular embodiment, the sequence of the IL7RA promoter used in the expression cassette of the invention comprises CpG islands capable of being demethylated under the action of Notch. For example, the IL7RA promoter sequence used in the expression cassette can comprise nucleotides −1100 to −300 of the human IL7RA gene (a region that comprises several methylation-sensitive CpG islands). In particular, the IL7RA promoter sequence used in the expression cassette can comprise the CpG islands in position −975, −567, −497, −475, −466 and −346 of the promoter of the human IL7RA gene. The IL7RA promoter sequence used in the expression cassette can further comprise the NF-kB binding site, located in position −187 to −173 of the human IL7RA gene. The IL7RA promoter sequence used in the expression cassette can further comprise the TATA box, in position −27 to −22 of the human IL7RA gene.

In some embodiments, the promoter sequence used in the expression cassette of the invention comprises:
a region of the native IL7RA promoter which comprises potential Notch binding domains, said region corresponding, for example, to nucleotides −927 to −920 of the human IL7RA gene; and
a region of the native IL7RA enhancer which comprises the potential Notch binding domain, said region corresponding, for example, to nucleotides +27755 to +27762 of the human IL7RA gene; and
one or more other region(s) from the promoter region of the human IL7RA gene.

In some embodiments, the sequence of the promoter used in the expression cassette according to the invention can also contain sequences of the IL7RA gene downstream of the transcription initiation site (downstream of nucleotide +1). For example, the sequence of the promoter used in the expression cassette can comprise nucleotides 1 to 10, or nucleotides 1 to +100, or nucleotides 1 to +200 of the IL7A gene. Thus, the promoter of the expression cassette can include a sequence (e.g., approximately 100, 200, 400, 600, 800, 1,000, 1,200, 1,400, 1,600 or 1,800 bp) within the region −1257 to +200 of the human IL7RA gene. In some embodiments, the promoter sequence size introduced into the expression cassette does not exceed approximately 2 kb (e.g., does not exceed 2.2 kb) so as to allow inclusion of a large coding sequence in the vector.

According to a preferred embodiment, the sequence of the IL7RA promoter introduced into the expression cassette according to the invention comprises or consists in a sequence comprising nucleotides −1257 to +89 of the IL7RA gene.

According to a particular embodiment, the IL7RA promoter sequence introduced into the expression cassette comprises or consists in the nucleic acid sequence SEQ ID NO: 1, or a functional variant having at least 80%, 85%, 90%, 95%, 96%, 97%, 98%, or at least 99% identity with the nucleic acid sequence SEQ ID NO: 1.

In the present invention, a “functional variant” of a promotor is a sequence derived from a parent promotor sequence, which has the same type of transcription initiation activity, but with a different nucleic sequence. Such a functional variant can have at least 70%, 75%, 80%, 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8% or at least 99.9% identity with the parent sequence. Such a functional variant leads to the same or substantially the same expression level of a given protein operably linked to the latter, as the promotor from which it is derived. For example, such a functional variant can lead to an expression level equivalent to at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 220%, 240%, 260%, 280%, 300%, 350%, 400%, 500%, or even at least 600%, relative to the level of expression obtained with the parent promoter from which it is derived.

The term “identical” and its variations refer to the sequence identity between two nucleic acid molecules. When a position in the two compared sequences is occupied by the same base, then the molecules are identical at that position. The percentage identity between two sequences is the number of identical positions between the two sequences divided by the number of positions compared×100. For example, if 6 of the 10 positions in two sequences match then the two sequences are 60% identical. Generally, a comparison is performed when two sequences are aligned to give maximum identity. Various bioinformatics tools known to the person skilled in the art can be used to align nucleic acid sequences such as BLAST or FASTA.

According to another preferred embodiment, the IL7RA promoter sequence introduced into the expression cassette according to the invention comprises:
a region comprising nucleotides −1257 to +89 of the IL7RA gene;
   and
a region comprising nucleotides +27,310 to +27,852 of the IL7RA gene.

According to a particular embodiment, the IL7RA promoter sequence comprises or consists in the nucleic acid sequence SEQ ID NO: 2, or a functional variant thereof having at least 80%, 85%, 90%, 95%, 96%, 97%, 98%, or at least 99% identity with the nucleic acid sequence SEQ ID NO: 2.

According to a particular embodiment, the IL7RA promoter sequence does not comprise the nucleic acid sequence SEQ ID NO: 2, or a functional variant thereof having at least 80%, 85%, 90%, 95%, 96%, 97%, 98%, or at least 99% identity with the nucleic acid sequence SEQ ID NO: 2. According to a particular embodiment, the IL7RA promoter sequence comprises the nucleic acid sequence SEQ ID NO: 1, or a functional variant thereof having at least 80%, 85%, 90%, 95%, 96%, 97%, 98%, or at least 99% identity with the nucleic acid sequence SEQ ID NO: 1, but does not comprise the nucleic sequence SEQ ID NO: 2, or a functional variant thereof having at least 80%, 85%, 90%, 95%, 96%, 97%, 98%, or at least 99% identity with the nucleic acid sequence SEQ ID NO: 2.

According to an embodiment, the IL7RA promoter sequence comprises or consists in the nucleic acid sequence SEQ ID NO: 1, or a functional variant thereof having at least 80%, 85%, 90%, 95%, 96%, 97%, 98%, or at least 99% identity with the nucleic acid sequence SEQ ID NO: 1, and comprises between 1300 and 1800 nucleotides, preferably between 1320 and 1700 nucleotides, preferably between 1340 and 1600 nucleotides, preferably between 1346 and 1500 nucleotides.

As used herein, a human IL7RA gene refers to an IL7RA gene from any human individual. It can contain genetic variations from the specific sequences mentioned here. The present disclosure considers all the functional equivalents of the promoter sequences specifically mentioned in the application.

The applicant has shown that the expression of a CAR transgene under the control of the extended IL7RA promoter (SEQ. 2) does not allow controlled expression of the transgene in a maturation-dependent manner, i.e. that the expression of the transgene remains stable during T maturation. The applicant also showed that expression under the control of this promoter is high in CD34+ hematopoietic stem cells. The use of this extended IL7RA promoter therefore allows stably elevated transgene expression in CD34+ haematopoietic stem cells. The applicant has therefore demonstrated that this extended promoter is a strong constitutive promoter in CD34+ hematopoietic stem cells, and therefore the possibility of using the IL7RA promoter comprising or consisting in the nucleic sequence SEQ ID NO: 2, or a functional variant thereof, for the modification of CD34+ hematopoietic stem cells and the expression of a heterologous nucleic acid sequence of interest in a stable and strong manner.

Thus, another aspect of the invention relates to an isolated T-cell progenitor, preferably human, having a CD45RA+, CD7+, CD5+ phenotype, said T-cell progenitor comprising an expression cassette which comprises the IL7RA promoter comprising or consisting in the nucleic sequence SEQ ID NO: 2, or a functional variant thereof having at least 80%, 85%, 90%, 95%, 96%, 97%, 98%, or at least 99% identity with the nucleic acid sequence SEQ ID NO: 2, the promoter being functionally linked to a heterologous nucleic acid sequence of interest.

The invention therefore also relates to an expression cassette comprising the IL7RA promoter region functionally linked to a heterologous nucleic acid sequence of interest, in which the IL7RA promoter region comprises or consists in a nucleic acid sequence having at least 85%, 90%, 95%, 96%, 97%, 98%, or at least 99% identity with the nucleic acid sequence SEQ ID NO: 2. According to a particular embodiment, the nucleic acid sequence of interest encodes a chimeric antigen receptor or a T cell receptor.

The invention also relates to a use of an expression cassette comprising the IL7RA or the BCL11B promoter region functionally linked to a heterologous nucleic acid sequence of interest, in which the IL7RA promoter region comprises or consists in a nucleic acid sequence having at least 85%, 90%, 95%, 96%, 97%, 98%, or at least 99% identity with the nucleic acid sequence SEQ ID NO: 2, or a vector comprising it, for the generation of T-cell progenitors.

Another aspect of the invention relates to a vector comprising an expression cassette as defined above. The vector is preferably a viral vector, preferably a retroviral vector, and more preferentially a lentiviral vector.

Alternatively, the expression cassette of the present description can comprise a promoter derived from the human BCL11B gene. In the present invention, a promoter “derived” from a human BCL11B gene refers to a promoter that contains a functionally active region of the promoter of a human BCL11B gene, or a functional equivalent thereof (e.g., a variant of the promoter region containing nucleotide changes relative to the native sequence where the nucleotide changes do not adversely affect the transcriptional regulatory activity of the promoter).

According to a particular embodiment, the sequence of the BCL11B promoter introduced into the expression cassette corresponds to a sequence located in the region of −4864 to +979 base pairs (relative to the transcription initiation site) of the BCL11B gene. The expression “nucleotides −4864 to +979” of a human BCL11B gene is understood to mean nucleotides −4864 to +979 of the human BCL11B gene as referenced in the NCBI database (NCBI reference: 64919), or Ensembl database (Ensembl reference: ENSG00000127152).

According to a particular embodiment, the BCL11B promoter sequence comprises or consists in the nucleic acid sequence SEQ ID NO: 3, or a functional variant thereof having at least 30%, 40%, 50%, 60%, 70%, 80%, 85%, 90%, 95%, 96%, 97%, 98%, or at least 99% identity with the nucleic acid sequence SEQ ID NO: 3.

According to a particular embodiment, said functional variant of the BCL11B promoter having at least 30%, 40%, 50%, 60%, 70%, 80%, 85%, 90%, 95%, 96%, 97%, 98%, or at least 99% identity with the nucleic sequence SEQ ID NO: 3, comprises a maximum of 4000 base pairs (bp), preferably a maximum of 3000 bp. For example, said functional variant can comprise from 500 to 4000 bp, preferably from 1000 to 3000 bp, preferably from 1000 to 2000 bp.

The ILR7A or BCL11B promoter is operably linked to a heterologous nucleic acid sequence of interest. The promoter sequence can be directly linked to the heterologous nucleic acid sequence of interest. Alternatively, the two sequences can be linked by a linking sequence.

As used herein, the expression “heterologous nucleic acid sequence of interest” refers to any sequence heterologous with respect to the promoter used. In particular, when the promoter is the IL7RA promoter, the nucleic sequence does not encode IL7RA. Similarly, if the promoter is the BCL11B promoter, the nucleic sequence does not encode BCL11B.

The heterologous nucleic acid sequence of interest can be any sequence having a therapeutic interest when expressed by T-cells.

For example, the invention can be used to produce additional copies of genes to allow increased expression by T cells of certain gene products in vivo.

The nucleic acid sequence of interest can be a nucleic acid sequence encoding a therapeutic protein or a therapeutic nucleic acid. The therapeutic protein or therapeutic nucleic acid when expressed by the T cell has a beneficial effect in treating a human or veterinary disease or disorder. The therapeutic nucleic acid is preferably a therapeutic RNA. The therapeutic protein can be a peptide or polypeptide.

Nucleic acid sequences of interest comprise, but are not limited to, those encoding a chimeric antigen receptor (CAR), a chimeric costimulatory receptor (CCR), a T-cell receptor (TCR), a cytokine, a hormone, an antibody, a biosensor, a soluble receptor, an enzyme, a ribozyme, reporter, an epigenetic modifier, a transcriptional activator or a repressor, a non-coding RNA, or the like.

It is understood that the nucleic acid sequence of interest can encode, for example, a cDNA, a gene, an miRNA or lncRNA, or other.

In a specific embodiment, the T-cell progenitors are modified to recognize an antigen such as a tumour antigen, a viral antigen, or a bacterial antigen. As such, the immune response to said targeted antigen will be enhanced by administration of antigen-specific T-cell progenitors.

According to a particular embodiment, the nucleic acid sequence of interest encodes a chimeric antigen receptor (CAR).

The present invention therefore relates in particular to an expression cassette comprising the IL7RA promoter region functionally linked to a heterologous nucleic acid sequence of interest, in which the nucleic acid sequence of interest encodes a chimeric antigen receptor (CAR) or a T cell receptor (TCR). According to one embodiment, the IL7RA promoter region comprises or consists in a nucleic acid sequence having at least 85%, 90%, 95%, 96%, 97%, 98%, or at least 99% identity with the nucleic acid sequence SEQ ID NO: 1 or SEQ ID NO: 2, preferably with the nucleic sequence SEQ ID NO: 1.

It also relates to an expression cassette comprising the BCL11B promoter functionally linked to a heterologous nucleic acid sequence of interest, in which the nucleic acid sequence of interest encodes a chimeric antigen receptor or a T cell receptor (TCR). According to one embodiment, the IL7RA promoter region comprises or consists in a nucleic acid sequence having at least 85%, 90%, 95%, 96%, 97%, 98%, or at least 99% identity with the nucleic acid sequence SEQ ID NO: 3.

A “chimeric antigen receptor (CAR)”, as used herein, refers to an artificial T cell receptor, and encompasses modified receptors that graft artificial specificity for a particular antigen onto an immune effector cell.

A CAR typically comprises an ectodomain (extracellular domain) and an endodomain (cytoplasmic domain), linked by a transmembrane domain. The ectodomain, expressed on the surface of the cell, comprises an antigen binding domain or receptor domain and, optionally, a spacer (or hinge) region linking the antigen binding domain to the transmembrane domain. The transmembrane domain is typically a hydrophobic alpha helix that extends through the lipid bilayer of the cell membrane. The CAR endodomain is composed of an intracellular signalling module that induces T-cell activation upon antigen binding. The endodomain can comprise a plurality of signalling domains.

In specific embodiments, the CAR can be a “first generation”, “second generation” or “third generation” CAR (see, for example, Sadelain et al., Cancer Discov. 3 (4): 388-398 (2013); Jensen et al., Immunol. Rev. 257:127-133 (2014); Sharpe et al., Dis. Model Mech. 8 (4): 337-350 (2015); Brentjens et al., Clin. Cancer Res. 13:5426-5435 (2007), Gade et al., Cancer Res). The role of the CAR signalling domain is to transduce the activation signal to the immune cell as soon as the extracellular domain has recognized the antigen. In most CAR designs implemented to date, CD3ζ signalling sequences are used as signalling domain triggering lytic activity. The first generation CARs contain only the CD3ζ chain. The second generation CARs comprise a cytoplasmic domain containing the CD28 costimulatory domain, fused with CD3ζ C. This design provides both an activation and proliferation signal to the T cell (consequently, the cell is activated, destroys the target cell and proliferates). In addition to CD28, costimulatory receptor signalling sequences such as CD134 (TNFRSF4, OX40), CD154 (CD40L), CD137 (4-1BB), ICOS (CD278), CD27 and CD244 (2B4), have been successfully tested in CARs. The third generation of CAR is based on the combination of two or more costimulatory sequences (such as 4-1BB-CD28-CD3ζ). These receptors secrete a wider range of cytokines (including TNFα, GM-CSF, and IFNγ). One or more endodomains can be used, since the so-called third generation CARs generally have at least 2 or 3 signalling domains fused for an additive or synergistic effect, for example.

In particular, the CAR has a binding domain that binds to an antigen, the antigen being associated with a disease or disorder present in the subject or desired to be prevented in the subject to which the cell is administered. In a particular embodiment, the CAR binds a tumour associated antigen (TAA).

In a particular embodiment, the antigen binding domain of the CAR is capable of binding a TAA selected from the following non-limiting list: CD19, CD20, ErbB2, MUC1, CD13, CD33, GD2, NCAM, ALK, CD52, CD160, CA-125, folate binding protein, CD5, EGFR, vimentin, BCMA, CD138, carbonic anhydrase IX, G250, PSMA, A33

In a particular embodiment, the antigen binding domain of the CAR is capable of binding TAA, as listed in the table below (non-exhaustive list).

TABLE 1

| Type of cancer | Tumour associated antigen (TAA) |
|---|---|
| Diffuse large B-cell lymphoma | CD19, CD20 |
| Breast cancer | ErbB2, MUC1 |
| Acute myeloid leukaemia | CD13, CD33 |
| Neuroblastoma | GD2, NCAM, ALK, |
| Chronic lymphocytic leukaemia | CD19, CD52, CD160 |
| Colorectal cancer | Folate binding protein, CA-125 |
| Chronic lymphocytic leukaemia | CD5, CD19, CD123 |
| Glioma | EGFR, vimentin |
| Multiple myeloma | BCMA, CD138 |
| Renal cell carcinoma | Carbonic anhydrase IX, G250 |
| Cancer of the prostate | PSMA |
| Cancer of the intestine | A33 |

In a particular embodiment, the antigen binding domain of the CAR is capable of binding to CD19.

A “T cell receptor (TCR)”, as used herein, denotes a molecule capable of recognizing a peptide when presented by an MHC molecule. The molecule can be a heterodimer of two chains a and β (or optionally γ and δ) or can be a single chain TCR construct. According to one embodiment, the TCR can be a hybrid TCR comprising sequences derived from more than one species. For example, it has been surprisingly found that murine TCRs are more efficiently expressed in human T cells than human TCRs. The TCR can therefore comprise human variable regions and murine constant regions.

TAA-specific TCRs can be easily generated by the person skilled in the art using any method known in the art.

For example, TAA-specific TCRs can be identified by the TCR gene capture method of Linnemann et al. (Nature Medicine 19, 1534-1541 (2013)). In short, this method uses a DNA-based high throughput strategy to identify TCR sequences by capturing and sequencing genomic DNA fragments encoding TCR genes and can be used to identify TAA-specific TCRs.

### Vector

Another aspect of the invention relates to a vector comprising an expression cassette as described above. The vector according to the invention can be any vector capable of transforming or transducing T-cell progenitors.

As used herein, the term “vector” refers to a nucleic acid molecule capable of transporting another nucleic acid to which it has been bound. A “vector” in the present invention comprises, but is not limited to, a viral vector, a plasmid, an RNA vector or a linear or circular DNA or RNA molecule which can consist of a chromosomal molecule, non-chromosomal molecule, semisynthetic nucleic acids or synthetic nucleic acids. The preferred vectors are those capable of autonomous replication (episomal vector) and expression of the nucleic acids to which they are bound (expression vectors). A large number of suitable vectors are known to the person skilled in the art and are commercially available. The expression cassette according to the invention can be cloned into a vector such as a plasmid, a phagemid, a cosmid, a phage derivative or a virus.

According to a particular embodiment, the vector according to the invention is a viral vector. Viral vectors comprise retroviruses including lentiviruses, adenoviruses, parvoviruses, adeno-associated viruses (AAV), coronaviruses, negative-stranded RNA viruses such as orthomyxoviruses (e.g., influenza virus), rhabdovirus (e.g., rabies virus and vesicular stomatitis virus), paramyxovirus (e.g., measles and Sendai), positive-stranded RNA viruses such as picornavirus and alphavirus, and double-stranded DNA viruses, including adenovirus, herpes virus (e.g. herpes simplex virus types 1 and 2, Epstein-Barr virus, cytomegalovirus) and poxvirus (e.g. vaccinia virus, avian pox virus and canary pox virus). Other viruses comprise Norwalk virus, togavirus, flavivirus, reovirus, papovavirus, hepadnavirus and hepatitis virus, for example. Examples of retroviruses include: avian sarcoma leukosis, mammalian type C, type B, type D, and HTLV-BLV group viruses, lentivirus and spumavirus.

A number of viral vectors have been described for gene transfer into mammalian cells. The viral vectors which can be used in the present invention comprise, but are not limited to, retroviruses, adenoviruses, adeno-associated viruses, herpesviruses and lentiviruses. Preferably, the vector is a lentiviral vector. In particular, the retrovirus can be chosen from Moloney murine leukaemia virus (MMLV), human immunodeficiency virus (HIV) or gibbon ape leukaemia virus (GALV).

As is known in the art, depending on the viral vector, additional appropriate sequences will be introduced into the expression cassette of the invention in order to obtain a functional viral vector. The appropriate sequences comprise ITRs for the AAV vectors, or LTRs for the lentiviral vectors. In this respect, the invention also relates to an expression cassette as described above, flanked by an ITR on each side, or flanked by an LTR on each side.

In some embodiments, the vector is a recombinant AAV vector whose genome comprises the expression cassette of the invention, flanked by AAV inverted terminal repeat (ITR) sequences at both ends. The AAV can be of any serotype, for example, AAV1, AAV2, AAV3, AAV4, AAV5, AAV6, AAV7, AAV8, AAV8.2, AAV9 or AAVrhlO. In another particular embodiment, the AAV vector is a pseudotyped vector, i.e., its genome and its capsid are derived from AAV of different serotypes.

Preferably, the vector according to the invention is a lentiviral vector. An advantage of lentiviral vectors is that they retain the ability to infect cells that do not divide, thereby increasing their ability to transduce a wide variety of cells, including quiescent cells. In particular, the lentiviral vector is derived from primate or non-primate lentiviruses. Examples of primate lentiviruses comprise human immunodeficiency virus (HIV), the causative agent of human acquired immunodeficiency syndrome (AIDS) and simian immunodeficiency virus (SIV). The non-primate lentiviral group comprises visna-maedi slow virus (VMV), caprine arthritis encephalitis virus (CAEV), equine infectious anaemia virus (EIAV), feline immunodeficiency virus (IVF), and bovine immunodeficiency virus (BIV). Preferably, the lentivirus is derived from the HIV virus.

The method for producing the vector, in particular the lentiviral vector, integrating the expression cassette is well known to the person skilled in the art.

By way of example, a lentiviral vector comprising in its genome the expression cassette of the invention can be produced using a conventional lentiviral system with 4 plasmids. Briefly, cells (e.g., HEK293T) are transfected with: (i) a plasmid comprising the expression cassette of the invention between a 3′LTR and a 5′LTR; (ii) a plasmid encoding HIV-1 gag/pol proteins; (iii) a plasmid encoding HIV-1 rev protein; and (iv) a plasmid encoding an envelope glycoprotein (e.g., VSV-G, vesicular stomatitis virus envelope glycoprotein).

Another aspect of the invention relates to a cell population enriched in modified T-cell progenitors, as described above. The term “enriched” means that the cell population comprises a proportion of T-cell progenitors greater than the proportion of any other cell type constituting the cell population. In other words, T-cell progenitors are the most represented cell type in the cell population.

According to a particular embodiment, the cell population comprises at least 40%, 50%, 60%, 70%, 80% of the said T-cell progenitors.

Preferably, the cell population comprises at least 90% of said T-cell progenitors, such as 91%, 92%, 93%, 94%, 95%, 96%, 97%, 98%, 99% or even 100%.

Negative and positive selection methods known in the art can be used for enriching T-cell progenitors according to the invention. For example, cells can be sorted based on cell surface antigens using fluorescence activated cell sorting (FACS), or magnetic cell sorting (after labelling a cell subpopulation with a magnetic particle that binds cells based on the presence of a cell surface antigen). Negative selection columns can be used to remove cells expressing specific surface markers.

Another aspect of the invention relates to the preparation of the modified T-cell progenitor and the cell population as described above.

The T-cell progenitor according to the invention is preferably derived from a stem cell. Stem cells can be obtained from any suitable source, including, without limitation, umbilical cord, blood, embryos, embryonic tissue, foetal tissue, bone marrow and thymus. In one embodiment, the stem cell is a hematopoietic stem cell (HSC). In another embodiment, the stem cell is an embryonic stem cell. In another embodiment, the stem cell is an induced pluripotent stem cell (IPSC).

In a preferred embodiment, the T-cell progenitors are derived from CD34+ hematopoietic stem cells, which are preferably derived from umbilical cord blood, peripheral blood or bone marrow. In a preferred embodiment, the CD34+ hematopoietic stem cells are derived from umbilical cord blood.

The stem cells used to generate the T-cell progenitors according to the invention can be obtained from the patient to be treated or from a donor.

In a preferred embodiment, the stem cells used to generate the T-cell progenitors of the invention are obtained from a healthy donor. The stem cells are preferably taken from the peripheral blood or bone marrow of the healthy donor. Donors usually receive injections of G-CSF to mobilize stem cells into the bloodstream for collection. Alternatively, stem cells can be harvested directly from the bone marrow of the healthy donor. According to a preferred embodiment, stem cells are collected from placental umbilical cord blood after delivery.

Typically, a sample from the patient or healthy donor is first depleted of non-stem cells or mature cells. Negative and positive selection methods known in the art can be used for enriching hematopoietic stem cells. For example, cells can be sorted based on cell surface antigens (such as CD34+) using fluorescence activated cell sorting (FACS), or magnetic cell sorting (after labelling a cell subpopulation with a magnetic particle that binds cells based on the presence of a cell surface antigen). Negative selection columns can be used to remove cells expressing specific surface markers.

The purification of CD34+ HSCs can, for example, be carried out by commercially available techniques using magnetic beads (CD34+ Microbead kit, human (Miltenyi Biotec), EasySep Human CD34 positive Selection Kit II (Stemcell Technologies)). The purity of the population enriched in CD34+ HSCs is preferably greater than 80%, 85% or 90%, even more preferably greater than 95%, such as 96%, 97%, 98%, 99% or 100%.

The stem cells are then grown under suitable conditions to generate T-cell progenitors according to the invention. In the invention, the differentiation of cells, i.e., the differentiation of strains into T-cell progenitors, is distinct from the maturation of T-cell progenitors into mature T cells.

The method thus comprises a step of growing CD34+ hematopoietic stem cells under conditions appropriate to generate T-cell progenitors according to the invention. In particular, CD34+ HSCs are grown under conditions appropriate to generate T-cell progenitors of phenotype:
CD45RA+, CD7+, CD5+, CD1a−, CD3−, CD4−, CD8− (T progenitors) or
CD45RA+, CD7+, CD5+, CD1a+, CD3−, CD4−, CD8− (T precursors).

According to a particular embodiment, the T-cell progenitors according to the invention have the CD45RA+, CD7+, CD5+ phenotype, preferably the CD45RA+, CD7+, CD5+, CD1a+ phenotype.

Preferably, the hematopoietic stem cells are grown in the presence of one or more Notch ligands (such as Delta-like-1 or Delta-like-4) for a time sufficient to form T-cell progenitors.

More preferentially, the stem cells are grown in the presence of cells expressing a Notch ligand. In a particular embodiment, CD34+ HSCs are grown in the presence of an OP9 cell line or fibroblasts expressing a Notch ligand, such as Delta-like-1 (DLL1) or Delta-like-4 (DLL4). In a preferred embodiment, CD34+ HSCs are grown in the presence of a murine stromal line OP9 expressing DLL4.

For example, the concentration of the HSCs in the culture is between 1×10² and 10⁹, preferably 1×10² to 1×10⁶, more preferably 1×10⁵ to 1×10⁶, preferably between 1×10⁵ and 5×10⁵. In a particular embodiment, the HSCs are inoculated at 100,000 cells per well and grown on a monolayer of OP9 cells expressing Delta-like-1 or 4 (OP9-DLL1 or OP9-DLL4).

One or more cytokines that promote cell engagement and differentiation into T-cell progenitors can also be used. The concentration of a cytokine in a culture is typically approximately 1 to 100 ng/mL. The following are representative examples of cytokines that can be used: fibroblast growth factor (FGF), including FGF-4 and FGF-2; stem cell factor (SCF) or kit-ligand (KL); Flt-31 (FMS-like tyrosine kinase 3 ligand) and interleukin-7 (IL-7). Preferably, the cytokines used are Flt-3-ligand and IL-7. Preferably, the culture medium is supplemented with SCF, Flt-3L and IL-7. In a particular embodiment, the CD34+ HSCs are grown in the presence of an OP9 line expressing DLL1 or DLL4, the culture medium being complemented with SCF, Flt-31 and/or IL-7.

The stem cells can be grown in a culture medium comprising conditioned or unconditioned medium. Examples of suitable media are: X-VIVO™ 10, X-VIVO™ 15, 20, MEMa, RPMI 1640, IMDM or DMEM. Preferably, the culture medium is MEMa medium. The culture medium can comprise serum (for example bovine serum, foetal bovine serum, bovine calf serum, horse serum, human serum or an artificial serum substitute) or it can be serum-free. Preferably, the serum is foetal bovine serum (FBS) or foetal calf serum (FCS). Preferably, the culture medium is supplemented with 20% FBS or FCS.

The culture conditions involve growing the HSCs for a period of time sufficient for the cells of the preparation to form T-cell progenitors. The cells are generally maintained in culture for 4 to 50 days, preferably 5 to 25 days, preferably 10 to 21 days. Preferably, the cells are maintained in culture for approximately 20 days. It will be understood that the cells can be maintained for the appropriate time required to obtain the desired cellular composition.

According to a preferred embodiment, the CD34+ HSCs are grown in the presence of cells expressing a Notch ligand (such as OP9-DLL1 or OP9-DLL4), in a culture medium supplemented with serum (such as FBS or FCS), cytokines (in particular IL-7 and/or Flt3L), and possibly SCF.

Consequently, the present application proposes a method of generating a T-cell progenitor comprising (a) growing a sample comprising stem cells or progenitor cells with cells which express a Notch ligand, optionally in the presence of cytokines and/or growth factors and (b) isolating/purifying the T-cell progenitors. The cells expressing a Notch ligand are preferably OP9 cells expressing DLL1 or DLL4. T-cell progenitors can be characterized by the phenotype:
CD45RA+, CD7+, CD5+, CD1a−, CD3−, CD4−, CD8− (T progenitors) or
CD45RA+, CD7+, CD5+, CD1a+, CD3−, CD4−, CD8− (T precursors).

According to a particular embodiment, the T-cell progenitors according to the invention have the CD45RA+, CD7+, CD5+ phenotype, preferably the CD45RA+, CD7+, CD5+, CD1a+ phenotype.

The method can comprise a T-cell progenitor enrichment step, for example by performing cell sorting by FACS or magnetic beads, based on surface antigens. According to a particular embodiment, the cell population is enriched so as to obtain at least 40%, 50%, 60%, 70%, 80%, preferably at least 90%, even more preferentially at least 95% of said T-cell progenitors.

According to a particular embodiment, the cell population is enriched so as to obtain at least 40%, 50%, 60%, 70%, 80%, preferably at least 90%, even more preferentially at least 95% of said T-cell progenitors exhibiting a phenotype:
CD45RA+, CD7+, CD5+, CD1a−, CD3−, CD4−, CD8− (T progenitors) or
CD45RA+, CD7+, CD5+, CD1a+, CD3−, CD4−, CD8− (T precursors).

According to a particular embodiment, the T-cell progenitors according to the invention have the CD45RA+, CD7+, CD5+ phenotype, preferably the CD45RA+, CD7+, CD5+, CD1a+ phenotype.

The method further comprises a step of gene modification of the cells in order to obtain T-cell progenitors which comprise the expression cassette as described above. Thus, the method further comprises a step of introducing the expression cassette as described above into the cell. The expression cassette of the invention can be introduced into the cells in the form of naked DNA or integrated into an appropriate vector.

The expression cassette can be introduced into the target cell by any known techniques such as chemical methods (for example, calcium phosphate transfection or lipofection using cationic lipids), non-chemical methods (for example, electroporation), particle-based methods (for example, magnetofection) and viral transduction using viral vectors such as adenoviral vectors, retroviral vectors, lentiviral vectors, adeno-associated viral (AAV) vectors, or hybrid viral vectors.

In a preferred embodiment, the method comprises a transduction step, by means of a viral vector comprising in its genome the expression cassette of the invention. In a particular embodiment, the viral vector is as described above.

The step of introducing the expression cassette can be performed before, during, or after the step of differentiating stem cells into T-cell progenitors.

As used in the present invention, the term “differentiation of stem cells into T-cell progenitors” means that the cells obtained after differentiation are no longer pluripotent but are still immature. Thus, the differentiation as described here does not concern thymic maturation which transforms immature T-cell progenitors into mature T cells.

In the present invention, differentiation takes place in vitro, while thymic maturation takes place in vivo, after administration, as applicable, of the modified T-cell progenitors to a subject.

According to one embodiment, the T-cell progenitor preparation method of the invention comprises the steps of:
a) growing CD34+ hematopoietic stem cells in a culture medium comprising a Notch ligand and cytokines for differentiating said CD34+ cells into T-cell progenitors having a CD45RA+, CD7+, CD5+ phenotype, preferably a CD45RA+, CD7+, CD5+, CD1a+ phenotype;
b) transforming or transducing of cells before, during or after said differentiation step (a) with a vector comprising an expression cassette as defined in the present invention.

In one embodiment, the Notch ligand is Delta-like-1 (DLL1) or Delta-like-4 (DLL4). In one embodiment of the invention, the CD34+ hematopoietic stem cells are preferably derived from umbilical cord blood, peripheral blood or bone marrow, more preferentially umbilical cord blood.

In particular, the step of introducing the expression cassette, preferably by transformation or transduction of a vector containing the expression cassette, can be carried out using non-differentiated CD34+ HSCs. According to this embodiment, the method of the invention comprises, in order, the steps of:
collection of stem cells and optionally purification, as described above;
gene modification by integration of the expression cassette, as described above; and
differentiation of genetically modified stem cells into T-cell progenitors according to the invention, as described above.

Thus, according to one embodiment, the T-cell progenitor preparation method of the invention comprises the steps of:
a) transforming or transducing CD34+ hematopoietic stem cells with a vector comprising an expression cassette as defined in the present invention;
b) growing the cells obtained in step a) in a culture medium comprising a Notch ligand and cytokines allowing the differentiation of said CD34+ cells into T-cell progenitors having a CD45RA+, CD7+, CD5+ phenotype, preferably a CD45RA+, CD7+, CD5+, CD1a+ phenotype.

According to another embodiment, the step of introducing the expression cassette, preferably by transformation or transduction of a vector containing the expression cassette, can be carried out during the step of differentiating the stem cells into T-cell progenitors. According to this embodiment, the method of the invention comprises, in order, the steps of:
collection of stem cells and optionally purification, as described above;
initiation of the differentiation of stem cells into T-cell progenitors, as described above.
gene modification by integration of the expression cassette, as described above; and
end of the step of differentiation of genetically modified cells into T-cell progenitors according to the invention.

Thus, according to one embodiment, the T-cell progenitor preparation method of the invention comprises the steps of:
a) growing CD34+ hematopoietic stem cells in a culture medium comprising a Notch ligand and cytokines for differentiating said CD34+ cells into T-cell progenitors having a CD45RA+, CD7+, CD5+ phenotype, preferably a CD45RA+, CD7+, CD5+, CD1a+ phenotype;
b) transforming or transducing cells during said differentiation step (a) with a vector comprising an expression cassette as defined in the present invention.

Thus, according to one embodiment, the step of differentiation of hematopoietic stem cells into T-cell progenitors continues after the transformation or transduction step.

In other words, according to one embodiment, the T-cell progenitor preparation method of the invention comprises the steps of:
a) growing CD34+ hematopoietic stem cells in a culture medium comprising a Notch ligand and cytokines for differentiating said CD34+ cells into T-cell progenitors having a CD45RA+, CD7+, CD5+ phenotype, preferably a CD45RA+, CD7+, CD5+, CD1a+ phenotype;
b) transforming or transducing cells with a vector comprising an expression cassette as defined in the present invention;
(c) differentiating of hematopoietic stem cells into modified T-cell progenitors.

According to this embodiment, the CD34+ HSCs can be grown on day 0 in the presence of cells expressing a Notch ligand such as OP9 cells expressing DLL1 or DLL4, in a culture medium preferably comprising cytokines (in particular IL7 and/or Flt3L), growth factors, and/or serum (such as FBS or FCS). The step of introducing the expression cassette, preferably by transformation or transduction of a vector containing the expression cassette, can be carried out during the differentiation step, for example between day 0 and day 18.

According to one embodiment, the transformation or transduction step can be carried out between day 4 and 18, preferably between day 6 and day 15, preferably between day 7 and day 10. The step of differentiating the stem cells into T-cell progenitors is then continued, the T-cell progenitors preferably being harvested/isolated from day 13, preferably from day 16.

According to one embodiment, the transformation or transduction step can be carried out between day 0 and day 4, preferably between day 0 and day 3, preferably between day 0 and day 2. According to one embodiment, the transformation or transduction step can be carried out on day 0. According to another embodiment, the transformation or transduction step can be carried out on day 1.

According to an alternative embodiment, the step of introducing the expression cassette, preferably by transformation or transduction of a vector containing the expression cassette, can be carried out during the step of differentiating the stem cells into T-cell progenitors. According to this embodiment, the method of the invention comprises, in order, the steps of:
collection of stem cells and optionally purification, as described above;
differentiation of genetically modified stem cells into T-cell progenitors, as described above;
gene modification of T-cell progenitors by integration of the expression cassette, as described above.

Thus, according to one embodiment, the T-cell progenitor preparation method of the invention comprises the following steps:
a) culture of CD34+ hematopoietic stem cells in a culture medium comprising a Notch ligand and cytokines for differentiating said CD34+ cells into T-cell progenitors having a CD45RA+, CD7+, CD5+ phenotype, preferably a CD45RA+, CD7+, CD5+, CD1a+ phenotype;
b) transformation or transduction of T-cell progenitors after said differentiation step (a) with a vector comprising an expression cassette as defined in the present invention.

The modified T-cell progenitors and the cell population enriched with T-cell progenitors according to the invention can be preserved until administration to the patient. For example, the cells or enriched cell population can be cryopreserved in a cryoprotective medium, such as a medium comprising human albumin and DMSO.

Another aspect of the invention relates to a pharmaceutical composition comprising the T-cell progenitor of the invention, or the population enriched in T-cell progenitors of the invention, in combination with a pharmaceutically acceptable vehicle.

The term “pharmaceutically acceptable vehicle” should be understood to mean any substance other than the active ingredient in a medicament. The pharmaceutically acceptable vehicle can be a diluent, adjuvant, excipient or vehicle with which the T-cell progenitor is administered. Its addition is intended to confer physicochemical and/or biochemical characteristics to promote the administration of T-cell progenitors, without altering the stability and efficiency of said cells.

The pharmaceutical composition can, for example, take the form of an aqueous solution comprising cells, in the form of a suspension or emulsion. The composition can typically be buffered to a selected pH. The composition can comprise a pharmaceutically acceptable carrier, e.g., water, saline, phosphate buffered saline, or any other carrier suitable for cell integrity and viability, and for administering the cellular composition to a subject, particularly a human. A person skilled in the art can easily determine the quantity of cells and optional additives in the composition of the invention.

The pharmaceutical composition is preferably isotonic, i.e. it has the same osmotic pressure as blood. Such isotonic formulations generally have an osmotic pressure from approximately 250 mOsm to approximately 350 mOsm. The desired isotonicity of the composition of the invention can be obtained by using sodium chloride or other pharmaceutically acceptable agents such as dextrose, boric acid, sodium tartrate or other inorganic or organic solutes.

According to a particular embodiment, the composition comprises at least two populations of T-cell progenitors according to the invention, in which each population of T-cell progenitors expresses a different heterologous nucleic sequence. For example, the composition can comprise a first population of T-cell progenitors that express a first CAR in a controlled manner, targeting a first antigen, in association with a second population of T-cell progenitors that express a second CAR targeting a second antigen in a controlled manner.

The composition can also contain, in addition to the T-cell progenitors according to the invention, one or more active ingredients(s) useful for the targeted indication or disease. Such active ingredients are present in combination with the T-cell progenitors according to the invention in quantities which are effective for the desired purpose.

The pharmaceutical composition according to the invention comprises an effective quantity of T-cell progenitors according to the invention, which can be determined according to the pathology, the patient (for example according to their weight or age) and/or the stage of the disease.

The administration of the cells or pharmaceutical composition can consist of administering from 10⁴ to 10⁹ cells per kg of bodyweight of the patient, preferably from 10⁵ to 10⁶ cells per kg of bodyweight of the patient. The cells or population of cells can be administered in one or more doses.

In another embodiment, the pharmaceutical composition comprises T-cell progenitors according to the invention, especially at least 100 cells, at least 200 cells, at least 400 cells, at least 500 cells, at least 700 cells, at least 1000 cells, at least 1500 cells, at least 2000 cells, at least 3000 cells, at least 5000 cells, at least 10,000 cells, at least 100,000 cells, at least 1 million cells, at least 10 million cells, or at least 100 million T-cell progenitors of the invention.

The pharmaceutical composition according to the invention can be stored by cryopreservation, for example in liquid nitrogen. According to this embodiment, the pharmaceutical composition can also comprise one or more cryoprotective agent(s). For example, the pharmaceutical composition can comprise a CS10 medium or any other medium containing serum and DMSO.

The pharmaceutical composition according to the invention can be formulated for any conventional route of administration including topical, enteral, oral, parenteral, intranasal, intravenous, intramuscular, subcutaneous or intraocular.

Preferably, the pharmaceutical composition according to the invention is administered enterally or parenterally. When administered parenterally, the pharmaceutical composition according to the invention is preferably administered intravenously.

The present invention also relates to the use of the expression cassette of the invention or of a vector comprising it, for the generation of T-cell progenitors.

In another embodiment, the expression cassette comprises the IL7RA or BCL11B promoter region functionally linked to a heterologous nucleic acid sequence of interest. In one embodiment, the IL7RA promoter region comprises or consists in a nucleic acid sequence having at least 85%, 90%, 95%, 96%, 97%, 98%, or at least 99% identity with the nucleic acid sequence SEQ ID NO: 1 or SEQ ID NO: 2, preferably with the nucleic sequence SEQ ID NO: 1. In one embodiment, the BCL11B promoter region comprises or consists in a nucleic acid sequence having at least 85%, 90%, 95%, 96%, 97%, 98%, or at least 99% identity with the nucleic acid sequence SEQ ID NO: 3.

In one embodiment, the expression cassette of the invention is used for the generation of T-cell progenitors according to the preparation method as described in the present invention.

The present invention relates to a T-cell progenitor, a population enriched with T-cell progenitors, or a composition comprising such cells, for use as a medicament or for use in treating a disease or disorder in a subject. The invention also relates to the use of a T-cell progenitor, a population enriched in T-cell progenitors or a composition comprising such cells in the manufacture of a medicament for treating a disease or disorder in a subject. Finally, it relates to a method of treating a disease or disorder in a subject comprising administering a therapeutically effective quantity of a pharmaceutical composition of the present invention.

As used herein, “treatment” refers to any action aimed at improving the health of patients. “Treatment” can include, but is not limited to, relieving or improving one or more symptoms, reducing the extent of the disease, stabilizing the condition of the disease (for example, maintaining a patient in remission), prevention of the disease or prevention of the spread of the disease, slowing the progression of the disease, and reducing the risk of the disease recurrence or remission (whether partial or total). A treatment can include curative or prophylactic effects. Desirable effects of treatment comprise decreased rate of disease progression, improvement or alleviation of disease status, and remission or improvement of prognosis. Relief can occur before signs or symptoms of the disease or condition appear, as well as after they appear. Thus, “treatment” can include the “prevention” of an undesirable disease or condition. In a particular embodiment, the treatment of a cancer comprises inhibiting the growth or proliferation of the cancer cells or destroying the cancer cells. In a particular embodiment, the treatment of cancer comprises reducing the risk or development of metastases. In another particular embodiment, the treatment of cancer can refer to prevention of relapse. Treating cancer can also refer to maintaining a subject in remission.

As used herein, the terms “disorder” or “disease” refer to an improperly functioning organ, part or structure of the body related to, for example, genetic or developmental factors, infection, nutritional deficiency or imbalance, toxicity, or adverse environmental factors. Preferably, these terms refer to a health disorder or disease, e.g., a disease that disrupts normal physical or mental functions. More preferably, the term “disease” refers to immune and/or inflammatory diseases that affect animals and/or humans. Preferably, the term “disease” refers to cancers, infectious diseases or immune diseases.

In a particular embodiment, the T-cell progenitor, population enriched with T-cell progenitors, or composition comprising such cells, is used in the treatment of a genetic disease, cancer, infectious disease, or immune disease such as innate immunodeficiency.

In a particular embodiment, the T-cell progenitor, the population enriched in T-cell progenitors, or the composition comprising said cells, is used in a diagnostic method. For example, the T-cell progenitor can be modified to express a CAR coupled to a fluorescent marker allowing the detection of a target of interest, and its use in a diagnostic method.

The term “genetic disease”, as used herein, refers to any disease related to the absence or alteration of gene expression.

The term “cancer” as used herein is defined as a disease characterized by the rapid and uncontrolled growth of aberrant cells. Cancer cells can spread locally or through the bloodstream and lymphatic system to other parts of the body.

The term “infectious diseases” refers to disorders caused by organisms, such as bacteria, viruses, fungi or parasites.

The term “immune disease” or “autoimmune disease”, as used herein, refers to a condition in a subject characterized by cell, tissue and/or organ damage caused by an immunological reaction of the subject to their own cells, tissues and/or organs. Immune diseases comprise acquired and innate immunodeficiencies.

The term “inflammatory disease” refers to a condition in a subject characterized by inflammation, such as chronic inflammation. Autoimmune disorders may or may not be associated with inflammation.

In one aspect, the disease or disorder to be treated is a condition selected from a proliferative disease or disorder, preferably cancer; an infectious disease or disorder, preferably a viral, bacterial or fungal infection; an inflammatory disease or disorder; and an immune disease or disorder, preferably autoimmunity or autoimmune diseases. Immune disorders comprise innate and acquired immunodeficiency such as HIV infection, severe combined immunodeficiency (SCID) or post-transplant treatment.

In a particular embodiment, the T-cell progenitor, the T-cell progenitor enriched cell population, and/or the pharmaceutical composition are useful for treating autoimmune disorders. Autoimmune disorders can affect almost all organs of the subject, including, but not limited to, the nervous, gastrointestinal and endocrine systems, as well as the skin and other connective tissues, eyes, blood and blood vessels.

In a particular embodiment, the T-cell progenitor, the T-cell progenitor enriched cell population and/or the pharmaceutical composition is for use in the treatment of cancer. Consequently, the present invention also relates to methods for inhibiting the growth of cancer in a subject in need thereof and/or preventing the formation of cancer and/or the spread of cancer in a subject in need thereof.

In a particular embodiment, the T-cell progenitor, the T-cell progenitor enriched cell population and/or the pharmaceutical composition is intended for use in preventing a relapse of a cancer.

In particular, the cancer is a solid tumour or hematopoietic cancer.

According to a particular embodiment, the cancer is a solid tumour.

Hematopoietic cancers are cancers of the blood or bone marrow. Examples of haematological cancers comprise leukaemias, including acute leukaemias (such as acute lymphoid leukaemia, acute myeloid leukaemia, acute myeloid leukaemia and myeloblastic, promyelocytic, myelomonocytic and monocytic leukaemias, erythroleukemia), chronic leukaemias (such as chronic myelocyte leukaemia, chronic myeloid leukaemia and chronic lymphocytic leukaemia), essential polycythemia, lymphoma, Hodgkin's disease, non-Hodgkin's lymphoma (indolent and high-grade forms), multiple myeloma, Waldenstrom's macroglobulinemia, heavy chain disease, myelodysplastic syndrome, hairy cell leukaemia and myelodysplasia.

In a preferred embodiment, the T-cell progenitors of the invention are used as a therapeutic product, ideally as a “ready-to-use” product.

As used herein, the term “subject” or “patient” refers to an animal, preferably a human mammal or a non-human mammal such as dogs, cats, horses, cows, pigs, sheep and non-human primates, among others. Preferably, the subject is a human. The human subject according to the invention can be a human being in the prenatal stage, a newborn, a child, an infant, an adolescent or an adult.

According to one embodiment, said T-cell progenitor, said cell population or said pharmaceutical composition is used in second-line treatment.

The present invention also relates to an in-vitro method for evaluating the thymic activity of a subject comprising the measurement in the sample of the quantity of recent thymic emigrant (RTE) cells, essential upstream of the administration of the cell population described above.

According to one embodiment, the sample is a blood sample, preferably a whole blood sample.

According to one embodiment, the quantity of RTE cells is the quantity of CD4 RTE cells, the quantity of CD8 RTE cells or the quantity of CD4 RTE cells and CD8 RTE cells.

According to one embodiment, the quantity of RTE cells can be measured by measuring at least the expression level of the CD31 marker on naïve T cells. According to a particular embodiment, the quantity of RTE cells can be measured by measuring the expression level of the CD31 marker and of one or more other markers among CD27, CCR7 and CD45RA. According to a particular embodiment, the quantity of RTE cells can be measured by measuring at least the level of expression of the markers CD27, CCR7, CD45RA and CD31.

According to one embodiment, the quantity of RTE cells is equivalent to the quantity of CD31high cells. According to one embodiment, the quantity of RTE cells is equivalent to the quantity of CD27, CCR7, CD45RA, CD31high cells.

The methods for measuring the expression of a marker are well known in the art and comprise, among others, labelling in flow cytometry according to standard methods.

Alternatively, the quantity of RTE cells can be measured by measuring the quantity of signal joint T-cell receptor excision circle (sjTREC), in particular by measuring the quantity of sjTREC DNA.

Methods for measuring the quantity of sjTREC, especially sjTREC DNA, are well known in the art and comprise, among others, quantification by PCR, qPCR or qRT-PCR.

In an advantageous embodiment, the presence of thymic activity in said sample makes it possible to envisage a therapeutic treatment with the modified T-cell progenitors of the invention. Indeed, the presence of thymic activity in said sample makes it possible to determine whether the subject is likely to respond to treatment with a pharmaceutical composition of the invention.

The present invention therefore also relates to an in-vitro method for determining whether a subject is likely to respond to treatment with a pharmaceutical composition of the invention, said method comprising:
a) measuring the quantity of recent thymic emigrant (RTE) cells in the sample,
b) comparing the quantity of RTE cells measured in the sample with a reference value,
c) determining whether the subject is likely to respond to treatment with a pharmaceutical composition of the invention, in which a quantity of RTE cells equal to or greater than the reference value indicates a likelihood to respond to treatment.

According to one embodiment, the reference value is the quantity of CD4 and/or CD8 cells in the sample derived from the subject. According to this embodiment, the subject is likely to respond to treatment with a pharmaceutical composition of the invention if the quantity of CD4 RTE cells is equal to or greater than 2% of the total quantity of naïve CD4 cells in the subject's sample, preferably equal to or greater than 4% of the total quantity of naïve CD4 cells in the subject's sample, preferably equal to or greater than 5% of the total quantity of naïve CD4 cells in the subject's sample, preferably equal to or greater than 8% of the total quantity of naïve CD4 cells in the subject's sample, preferably equal to or greater than 10% of the total quantity of naïve CD4 cells in the subject's sample. According to another embodiment, the subject is likely to respond to treatment with a pharmaceutical composition of the invention if the quantity of CD8 RTE cells is equal to or greater than 10% of the total quantity of naïve CD8 cells in the subject's sample, preferably equal to or greater than 15% of the total quantity of naïve CD8 cells in the subject's sample, preferably equal to or greater than 20% of the total quantity of naïve CD8 cells in the subject's sample, preferably equal to or greater than 25% of the total quantity of naïve CD8 cells in the subject's sample, preferably equal to or greater than 30% of the total quantity of naïve CD8 cells in the subject's sample, preferably equal to or greater than 35% of the total quantity of naïve CD8 cells in the subject's sample.

According to one embodiment, the reference value is the quantity of RTE cells in a control sample derived from one or more healthy subjects, i.e. subjects with normal thymic activity. According to this embodiment, the subject is likely to respond to treatment with a pharmaceutical composition of the invention if the quantity of CD4 RTE cells is equal to or greater than 10% of the quantity of CD4 RTE cells in the control sample, preferably equal to or greater than 15% of the quantity of CD4 RTE cells in the control sample, preferably equal to or greater than 20% of the quantity of CD4 RTE cells in the control sample, preferably equal to or greater than 25% of the quantity of CD4 RTE cells in the control sample, preferably equal to or greater than 30% of the quantity of CD4 RTE cells in the control sample. According to another embodiment, the subject is likely to respond to treatment with a pharmaceutical composition of the invention if the quantity of CD8 RTE cells is equal to or greater than 10% of the quantity of CD8 RTE cells in the control sample, preferably equal to or greater than 15% of the quantity of CD8 RTE cells in the control sample, preferably equal to or greater than 20% of the quantity of CD8 RTE cells in the control sample, preferably equal to or greater than 25% of the quantity of CD8 cells in the control sample, preferably equal to or greater than 30% of the quantity of CD8 RTE cells in the control sample.

According to one embodiment, the reference value is the level of expression of the CD31 marker in the naïve CD4 and/or CD8 cells in the sample derived from the subject. According to one embodiment, the reference value is the level of expression of the CD31 marker in naïve CD4 and/or CD8 cells in a control sample derived from one or more healthy subjects, i.e. subjects with normal thymic activity.

According to one embodiment, the reference value is the level of expression of the CD31 marker in naïve CD4 and/or CD8 cells in a control sample derived from one or more healthy subjects, i.e. subjects with normal thymic activity. According to this embodiment, the subject is capable of responding to treatment with a pharmaceutical composition of the invention if the level of expression of the CD31 marker in the naïve CD4 cells is equal to or greater than 10%, 15%, 20%, 25% or 30% of the CD31 marker expression level in naïve CD4 cells of the control sample. According to another embodiment, the subject is capable of responding to treatment with a pharmaceutical composition of the invention if the level of expression of the CD31 marker in the naïve CD8 cells is equal to or greater than 10%, 15%, 20%, 25% or 30% of the CD31 marker expression level in naïve CD8 cells of the control sample.

According to another embodiment, the reference value is the quantity of sjTREC DNA in a control sample derived from one or more healthy subjects, i.e. subjects with normal thymic activity.

The present invention further relates to a method of treating a disease or disorder in a subject comprising an in-vitro method of determining whether a subject is likely to respond to the treatment of the present invention, and administering a pharmaceutical composition as described in the present invention.

According to one embodiment, the subject is at risk or is likely not to respond to treatment with the pharmaceutical composition of the present invention. According to this embodiment, the method therefore comprises a step of determining the subject capable of responding to said treatment.

The present invention further relates to a method of treating a disease or disorder in a subject comprising:
a) determining whether the subject is likely to respond to such treatment by in-vitro evaluation of thymic activity in a blood sample of the subject, in particular by:
   measuring the quantity of recent thymic emigrant (RTE) cells in the sample,
   comparing the quantity of RTE cells measured in the sample with a reference value,
   determining whether the subject is likely to respond to treatment with a pharmaceutical composition of the invention, in which a quantity of RTE cells equal to or greater than the reference value indicates a likelihood to respond to treatment.
b) administering to the subject likely to treatment a therapeutically effective quantity of a pharmaceutical composition of the present invention.

### EXAMPLES

The inventors have developed a T-cell progenitor expressing a nucleic acid sequence of interest in a controlled manner. More particularly, the nucleic acid sequence of interest is placed under the control of an exogenous promoter induced by the Notch pathway, thus allowing controlled expression according to the maturation state of the T-cell progenitors.

Experiments were performed with the IL7RA gene promoter (also known as the “IL7R” gene). The gene sequence of the IL7RA gene promoter encoding the IL7R alpha chain is shown in FIG. 1. The sequence from the NCBI database #3575 is used as the reference sequence. The start codon “ATG” for initiation of protein synthesis is in position +90 of the sequence. The transcription factors GFI-1 and GABPa bind to 5′-TAAATCAC[AT]GCA-3′ sequences and purine-rich repeats (GA repeats), respectively. The promoter also contains CpG methylation islands in positions-975, −567, −497, −475, −466 and −346. The promoter comprises the NF-kB binding site, located in position −187 to −173 of the human IL7RA gene. The promoter comprises the TATA box, in position −27 to −22 of the human IL7RA gene.

Two different sequences corresponding to the IL7RA promoter were tested in these experiments: a so-called “restricted” sequence named “SEQ. 1” (SEQ ID NO: 1) and an extended sequence named “SEQ. 2” (SEQ ID NO: 2).

### Example 1: Expression of IL7RA is Induced Via the Notch Pathway in Lymphoid Cells

### Materials and Methods

The cell lines used are Jurkat (immature T cells) and THP-1 (a non-lymphoid T cell line). The Jurkat or THP-1 cells are inoculated at 100,000 cells per well in an RPMI medium (Thermo Fisher Scientific) containing 10% of foetal bovine serum (Thermo Fisher Scientific) and 1% of penicillin-streptomycin (Thermo Fisher Scientific) containing or not containing Delta-like 4 Notch ligand. After a stimulation of 4 days, the cells are recovered in dry pellets. Molecular analysis of the mRNA expression of IL7RA and HES (control) is carried out by RT-qPCR according to the manufacturers' standard protocols. Total RNA is isolated with TRIzol (Thermo Fisher Scientific) according to the standard procedure. The reverse transcription (RT) reaction is carried out using SuperScript VILO™ cDNA synthesis kit (Thermo Fisher Scientific) and the quantitative PCR (qPCR) is carried out with PowerUp SYBR Green Master Mix (Thermo Fisher Scientific) on a Light Cycler 480 (Roche). The primer sequences used are as follows: IL7RA-sense 5′-TCGCTCTGTTGGTCATCTTG-3′, IL7RA-antisense 5′-GGAGACTGGGCCATACGATA-3′, S14-sense 5′-GGCAGACCGAGATGAATCCTCA-3′, S14-antisense 5′-CAGGTCCAGGGGTCTTGGTCC-3′, HES-sense 5′-AAAAATTCCTCGTCCCCGGT-3′, HES-antisense 5′-GGCTTTGATGACTTTCTGTGCT-3′. The expression of the mRNA is calculated according to the method of 2^{-ΔCt}, where ΔCt-Ct_{target gene}−CT_{housekeeping gene (S14)}.

### Results:

The results of FIG. 2 demonstrate that stimulation of the Notch pathway by its ligand DLL4 induces the expression of the HES reporter gene in the lymphoid T (Jurkat) and non-lymphoid (THP-1) lines. In return, the expression of IL7RA mRNA is induced only in the lymphoid T line. This clearly demonstrates that the endogenous expression of IL7RA under the control of its promoter is induced by the Notch pathway only in lymphoid populations.

### Example 2: Expression of the GFP Transgene Under the Control of the IL7RA Promoter

### Materials and Methods

The IL7RA promoter sequences (restricted SEQ. 1 and extended SEQ. 2) are synthesized de novo (GeneArt Gene Synthesis Service, Thermo Fisher Scientific) and cloned into a lentiviral vector pRLSIN.cPPT.PGK-GFP.WPRE (Addgene, catalogue #12252) by the conventional digestion/ligation techniques used in molecular biology.

The cell lines used are Jurkat (immature T cells) and THP-1 (a non-lymphoid T cell line). The Jurkat or THP-1 cells are inoculated at 500,000 cells per well in an RPMI medium (Thermo Fisher Scientific) containing 10% of foetal bovine serum (Thermo Fisher Scientific) and 1% of penicillin-streptomycin (Thermo Fisher Scientific) and transduced by adding the viral supernatant at 1×10⁶ TU/mL. After transduction for 6 hours, the cells are washed 2 times with PBS (Thermo Fisher Scientific) and resuspended in complete RPMI medium. After 7 days, the expression of the green fluorescent protein (GFP) marker is analysed by flow cytometry. The samples are acquired on an LSRII Fortessa cytometer (Becton Dickinson). The data are analysed with FlowJo v10.7.1 (BD Biosciences). Molecular analysis of the expression of GFP mRNA (control) is carried out according to the protocol described above.

### Results:

The introduction of the exogenous transgene (GFP) under the control of the IL7RA promoter (SEQ. 1 and 2) allows high expression in Jurkat cells. Moreover, the results of the qPCR show that stimulation with DLL4 of these cells tends to increase this expression further (FIG. 3.B). Expression of GFP under the control of the constitutive promoter is elevated in THP-1 cells. In contrast, the expression of GFP is low for the construction with the IL7RA promoter SEQ. 1 and 2. The FACS data are confirmed in qPCR (FIG. 3.C). This demonstrates that expression of the exogenous transgene under the control of the IL7RA promoter is induced by the Notch pathway only in lymphoid populations.

### Example 3: Expression of the CAR Transgene Under the Control of the IL7RA Promoter

### Materials and Methods

The transgene corresponding to the chimeric antigen receptor (CAR) molecule is cloned under the control of the IL7RA promoter. The CAR construct corresponds to the combination of the following elements: scFv of the anti-human CD19 antibody (clone FMC63), an intracellular activation domain 41BB and CD3 zeta (third generation CAR, GeneArt Gene Synthesis Service, Thermo Fisher Scientific). This sequence is introduced into the vector described above by the conventional digestion/ligation techniques used in molecular biology. The THP-1 cell line is transduced and the expression of the transgene is analysed as described above.

### Results

The results of FIG. 4 correspond to the expression of a CAR-GFP under the control of the IL7RA promoter (SEQ. 1 and 2) in the non-lymphoid line THP-1. The results show a drastic decrease in GFP expression when the IL7RA promoter (SEQ. 1) is used in non-lymphoid THP-1 cells. These results are in agreement with the results of qPCR (FIG. 2.C) where the expression of the endogenous IL7RA gene under the control of its promoter was not induced during the stimulation of the Notch pathway by its DLL4 ligand.

### Example 4: The IL7RA Promoter is a Strong Promoter

### Materials and Methods

A lentiviral vector having the extended sequence SEQ. 2 from the promoter to IL7RA or a control vector with a strong spleen focus-forming virus (SFFV) promoter are used to transduce the Jurkat cell line as described above. On day 7 post-transduction, the percentage of transduction is >60% for the presence of a green fluorescent protein (GFP) marker. The samples are acquired on an LSRII Fortessa cytometer (Becton Dickinson). The data are analysed with FlowJo v10.7.1 (BD Biosciences). The mean fluorescent intensity (MFI) is indicated on each graph of the samples acquired simultaneously.

### Results

It is known that the use of strong promoters (SFFV, EF1a) is preferred for lentiviral transduction of hematopoietic stem cells (CD34+ HSC) and induced pluripotent stem cells (iPSC). FIG. 5 shows here that the IL7RA promoter is a strong promoter, because the expression of the GFP transgene in transduced Jurkat lymphoid cells has the same intensity (same mean fluorescence intensity (MFI)) and same frequency (same percentage of GFP) as in cells transduced with the control vector (SFFV promoter).

### Example 5: Expression of IL7RA is Induced Via the Notch Pathway in Haematopoietic Stem Cells

### Materials and Methods

CD34+ hematopoietic stem cells are isolated from umbilical cord blood. Briefly, the mononuclear cells of the blood are separated by a Ficoll-Paque gradient (Cytiva Ficoll-Paque™, Fisher Scientific). The CD34+ cells are isolated by magnetic beads according to the supplier's protocol (ultrapure CD34 kit, Miltenyi Biotec). The purity of the enriched population is >95%. The cells thus isolated are exposed or not exposed to the Notch ligand (DLL4) in the culture medium for 6 days (FIG. 6.A). Flow cytometry analysis is performed after extracellular labelling with anti-CD127-V450 antibody (BD Biosciences) according to the standard labelling protocol. The samples are acquired on an LSRII Fortessa cytometer (Becton Dickinson). The data are analysed with FlowJo v10.7.1 (BD Biosciences). Molecular analysis of the expression of IL7RA and HES (control) mRNA is carried out according to the protocol described above.

### Results

In haematopoietic stem cells (CD34+ HSC) activation of the Notch pathway, a key pathway for T-lymphopoiesis, induces expression of IL7R protein (FIG. 6.B) and HES mRNA (control) and IL7RA mRNA (FIG. 6.C). Thus, IL7RA expression is induced via activation of the Notch pathway following interaction with its DLL-4 ligand in haematopoietic stem cells.

### Example 6: Transgene Expression Under IL7RA Promoter Control During T Differentiation In Vitro

### Materials and Methods

The CD34+ hematopoietic stem cells are purified as described above. On day 0, CD34+ cells are purified and grown at 500,000 per well in a plate containing OP9-DLL1 feeder stromal cells in T differentiation medium composed of: MEMa medium containing foetal bovine serum (20% FBS, Cytiva HyClone™ foetal bovine serum (US), defined) and 1% penicillin-streptomycin (Thermo Fisher Scientific). On day 0 a cocktail of cytokines necessary for their activation is added: IL-7, Flt3L, SCF, TPO (100 ng/mL, Bio-Techne). After activation, the cells are transduced by adding the lentiviral supernatant (SFR BioSciences Gerland-Lyon Sud (UMS3444/US8) to MOI between 20 and 50. The transduction agent is Protransduzin™ (at 15 μg/mL, JPT Peptides). The vector and the transduction agent are prepared according to the supplier's protocol. 24 hours post-transduction the medium is changed to fresh medium containing IL-7, Flt3L (5 ng/ml, Bio-Techne). Half of the medium is removed twice a week and fresh medium containing twice the cytokine concentration is added. At different days of co-culture, the populations of interest are analysed by flow cytometry: T precursors (CD45RA+CD7+, “pre-T”), CD3-negative (CD4−CD8−CD3−, “CD3−”) and CD3−positive (CD4−CD8−CD3+, “CD3+”). The antibodies used are all from BD Biosciences: CD1a-phycoerythrin (PE), CD45RA-PE-TexasRed, CD7-PE-Cy7, CD4-Pacific Blue, CD3-Alexa Fluor 700, CD5-ALLOPHYCOCYANIN (APC), CD8-APC-H7. The labelling protocols are standard. The samples are acquired on an LSRII Fortessa cytometer (Becton Dickinson). The data are analysed with FlowJo v10.7.1 (BD Biosciences).

### Results

The expression of the transgene under the control of the constitutive promoter (cntrl) is on average 12.38% and does not vary during T maturation in vitro (mean 12.63 and 12% for CD3− and CD3+ stages respectively) (FIG. 7). In return, expression of the CAR transgene under the control of the IL7RA promoter (SEQ. 1) is on average 15.3% at the early stage of pre-T, concomitant with Notch signalling, decreases in CD3− populations (3.73%) and increases in the most mature CD3+ populations (5.7%). The expression of the CAR transgene under the control of the IL7RA promoter (SEQ. 2) does not allow controlled expression of the transgene (we previously observed that this promoter had the “leaky” profile, the expression of the transgene remains stable during T maturation (15.15% for the pre-T stage, 16.8% for the CD3− stage and 19.5% for the CD3+ stage). These results demonstrate that the expression of the transgene under the control of the IL7RA promoter varies according to the stage of maturation of the T cells.

### Example 7: Expression of Endogenous IL7RA Gene and Transgene Under IL7RA Promoter Control

### Materials and Methods

Naïve CD4+ T cells are isolated from umbilical cord blood. Briefly, the mononuclear cells of the blood are separated by a Ficoll-Paque gradient (Cytiva Ficoll-Paque™, Fisher Scientific). The naïve CD4+ cells are isolated by magnetic beads according to the supplier's protocol (naive CD4 T-cell isolation kit, Miltenyi Biotec). The purity of the enriched population is >97%. The cells are then transduced according to the procedure described above and are stimulated or not by Notch ligand DLL4. After 6 days post-transduction, the expression of endogenous IL7RA is measured by flow cytometry, as well as the expression of the transgene (based on GFP). The labelling protocols are standard. The samples are acquired on an LSRII Fortessa cytometer (Becton Dickinson). The data are analysed with FlowJo v10.7.1 (BD Biosciences).

### Results

The expression of the endogenous IL7RA protein does not vary in the absence or presence of DLL4 (ligand activating the Notch pathway) (FIG. 8).

Similarly, the expression of the transgene under the control of the control vector and of the vectors containing the sequence of the CAR transgene under the control of the IL7RA promoter (SEQ. 1 and SEQ. 2) is not different in the absence or presence of DLL4. This suggests that induction of transgene expression under the control of the IL7RA promoter (SEQ. 1) is cyclic and finely controlled in haematopoietic stem cells during T differentiation via the Notch pathway. In return, this regulation is no longer ensured via the Notch pathway in mature T cells, thus allowing a constant expression of the transgene in mature T cells via the transcription factors described such as GFI-1 and GABPalpha.

### Conclusions

The experiments conducted demonstrate that the use of the IL7RA promoter regulates the expression of a transgene of interest during T cell maturation stages from hematopoietic stem cells (CD34+ HSCs). More particularly, this fine regulation of the expression of the transgene thus makes it possible to avoid the depletion of transgenic T cells during thymic selection steps while allowing the expression of the transgene of interest once the T cells are mature. This has a considerable advantage when considering cell and gene therapies based on immature T cells which, once injected into the patient, are required to progress through the stages of maturation to acquire immunocompetence and polyfunctionality. An allogeneic CAR-T therapy in the field of oncology is one of the possible applications of the invention. The immature aspect of the cells makes it possible to envisage their use in a context where the donor and the recipient are two different people. The regulation of the expression of the maturation-dependent molecule through the use of the IL7RA promoter will thus provide the transgenic cells with a selective advantage during the negative selection which will take place in vivo in the patient. This advantage should be confirmed in the patient, since, to date, there are no relevant in-vitro models to summarize the complex steps of positive and negative selections.

### Example 8: Non-Invasive Measurement of Thymic Activity

### Materials and Methods

Whole blood samples from healthy donors (HD, n=5), untreated (UT, n=4) and treated (TT, n=21) leukaemia or lymphoma patients were collected. They were then treated for labelling in flow cytometry according to the standard method. Labelling was performed with the following antibodies: CD4-Pacific Blue, CD3-Alexa Fluor 700, CD8-APC-H7, CCR7-Alexa Fluor 647 (BD Biosciences), CD45RA-PE-TexasRed, CD31-PE (Miltenyi Biotech) and CD27-PE-Cy7 (eBioscience), LIVE/DEAD aqua fluorescent dye (Invitrogen). The samples were acquired on an LSRII Fortessa cytometer (Becton Dickinson). The data were analysed with the FlowJo v10.7.1 software (BD Biosciences).

The quantity of RTE cells was measured by quantifying CD4 CD27+CCR7+CD45RA+CD31high and CD8 CD27+CCR7+CD45RA+CD31high cells following the flow cytometry labelling described above. The data analysis was carried out using GraphPad Prism software, the statistical test used was the non-parametric Kruskal-Wallis test with a Dunn's post-hoc test. The mean age of the patients is shown in the table below (Table 2).

TABLE 2

| Patient | Type | Mean age | |
|---|---|---|---|
| HD | Healthy | +/−47.5 | years |
| UT | Leukaemia/lymphoma without treatment | +/−24.5 | years |
| TT | Leukaemia/lymphoma after chemotherapy | +/−24 | years |

### Results

The CD27+CCR7+CD45RA+CD31high population represents the recent thymic emigrants (RTE) which are the naïve T cells expressing the CD31high marker testifying to their recent emigration from the thymus. Once at the periphery, at the first contact with an antigen, this marker is down-regulated and is no longer re-expressed. It has been shown that the frequency of RTE is correlated with the quantity of sjTREC DNA. SjTREC DNA is the result of the rearrangement of TCR receptor chains and is a measure of T lymphopoiesis (thymic activity). It has been shown that as an individual ages, thymic activity decreases drastically in healthy subjects (V Geenen et al., J of Endocrinology (2003) 176, 305-311).

FIG. 9A shows an example of CD4, CD8, CD3, CCR7, CD45RA, CD31 and CD27 of a whole blood sample. The quantity of CD4 CD31high or CD8 CD31high cells compared to the quantity of CD4 or CD8 cells determines the quantity of RTE cells in the sample.

The results show that the untreated patients (UT patients) statistically have the same thymic activity as the healthy donors (HD) (FIG. 9B). On the other hand, the treated patients (TT patients) have, on average, a lower thymic activity than the healthy donors (FIG. 9B). More precisely, treated patients can be divided into 2 groups: those with thymic activity and those without thymic activity or with very low activity (FIG. 9C and Table 3).

TABLE 3

| | HD | UT | TT | TT |
|---|---|---|---|---|
| Treatment | − | − | + | + |
| Activity | + | + | + | − |

This test therefore makes it possible to select eligible patients (i.e., having thymic activity) for the application of allogeneic CAR-T therapies as described in the present patent application.

1-16. (canceled)

17. An isolated T-cell progenitor having a CD45RA+, CD7+, CD5+ phenotype, the T-cell progenitor comprising an expression cassette which comprises an exogenous promoter induced by the Notch pathway functionally linked to a heterologous nucleic acid sequence of interest,
wherein the exogenous promoter is chosen between the IL7RA and the BCL11B promoter.

18. The isolated T-cell progenitor of claim 17, wherein the T-cell progenitor is human.

19. The isolated T-cell progenitor of claim 17, wherein the T-cell progenitor has a CD45RA+, CD7+, CD5+CD1a+ phenotype.

20. The isolated T-cell progenitor of claim 17, wherein the heterologous nucleic acid sequence of interest encodes a chimeric antigen receptor.

21. A cell population comprising the T-cell progenitors of claim 17, the cell population preferably comprising at least 40% of the T-cell progenitors.

22. A pharmaceutical composition comprising the T-cell progenitor of claim 17.

23. A pharmaceutical composition comprising the cell population of claim 21.

24. An expression cassette comprising the IL7RA promoter functionally linked to a heterologous nucleic acid sequence of interest, wherein the nucleic acid sequence of interest encodes a chimeric antigen receptor or a T cell receptor.

25. The expression cassette of claim 24, wherein the IL7RA promoter region comprises or consists in a nucleic acid sequence having at least 85% identity with the nucleic acid sequence SEQ ID NO: 1 or the nucleic acid sequence SEQ ID NO: 2.

26. An expression cassette comprising the BCL11B promoter functionally linked to a heterologous nucleic acid sequence of interest, wherein the nucleic acid sequence of interest encodes a chimeric antigen receptor or a T cell receptor.

27. The expression cassette according to claim 8, wherein the BCL11B promoter comprises or consists in a nucleic acid sequence having at least 85% identity with the nucleic acid sequence SEQ ID NO: 3.

28. A vector comprising the expression cassette of claim 24.

29. The vector of claim 28, wherein the vector is a viral vector.

30. The vector of claim 29, wherein the vector is a retroviral vector.

31. The vector of claim 30, wherein the vector is a lentiviral vector.

32. A method of preparing the T-cell progenitor of claim 1, comprising the steps of:
a) growingCD34+ hematopoietic stem cells in a culture medium comprising a Notch ligand such as Delta-like-1 (DLL1) or Delta-like-4 (DLL4) and cytokines for differentiating said CD34+ cells into T-cell progenitors having a CD45RA+, CD7+, CD5+ phenotype, preferably a CD45RA+, CD7+, CD5+, CD1a+ phenotype;
b) transforming or transducing cells before, during or after said differentiation step (a) with a vector comprising an expression cassette,
the expression cassette comprising an exogenous promoter induced by the Notch pathway functionally linked to a heterologous nucleic acid sequence of interest,
the exogenous promoter being chosen between the IL7RA and the BCL11B promoter, and
the nucleic acid sequence of interest encoding a chimeric antigen receptor or a T cell receptor; and
wherein the CD34+ hematopoietic stem cells are derived from umbilical cord blood, peripheral blood or bone marrow.

33. The method of claim 32, wherein the CD34+ hematopoietic stem cells are derived from umbilical cord blood.

34. A method of treatment of a cancer, an infectious disease, a genetic disease, an inflammatory disease or an immune or autoimmune disease comprising the administration of a T-cell progenitor to a subject in need thereof,
the T-cell progenitor having a CD45RA+, CD7+, CD5+ phenotype and comprising an expression cassette which comprises an exogenous promoter induced by the Notch pathway functionally linked to a heterologous nucleic acid sequence of interest,
wherein the exogenous promoter is chosen between the IL7RA and the BCL11B promoter.

35. The method of claim 34, wherein the T-cell progenitor is autologous or allogeneic with respect to the patient to be treated.

36. The method of claim 34, wherein the T-cell progenitor is allogeneic with respect to the patient to be treated.
